SEQUENCE LISTING (25 sequences)

SEQ ID NO: 1 - DNA, 194 bp, Artificial Sequence
  Description of Artificial Sequencepromoter sequence
gctattgacgacagctatggttcactgtccaccaaccaaaactgtgctcagtaccgccaatatttctcccttgaggggtacaaagaggtgtccctagaagagatccacgctgtgtaaaaattttacaaaaaggtattgactttccctacagggtgtgtaataatttaattacaggcgggggcaaccccgcctgt

SEQ ID NO: 2 - DNA, 163 bp, Artificial Sequence
  Description of Artificial Sequencepromoter sequence
gcctacctagcttccaagaaagatatcctaacagcacaagagcggaaagatgttttgttctacatccagaacaacctctgctaaaattcctgaaaaattttgcaaaaagttgttgactttatctacaaggtgtggtataataatcttaacaacagcaggacgc

SEQ ID NO: 3 - DNA, 127 bp, Artificial Sequence
  Description of Artificial Sequencepromoter sequence
gaggaatcatagaattttgtcaaaataattttattgacaacgtcttattaacgttgatataatttaaattttatttgacaaaaatgggctcgtgttgtacaataaatgtagtgaggtggatgcaatg

SEQ ID NO: 4 - DNA, 24 bp, Artificial Sequence
  Description of Artificial Sequenceribosome binding site
taaacatgaggaggagaaaacatg

SEQ ID NO: 5 - DNA, 28 bp, Artificial Sequence
  Description of Artificial Sequenceribosome binding site
attcgagaaatggagagaatataatatg

SEQ ID NO: 6 - DNA, 13 bp, Artificial Sequence
  Description of Artificial Sequenceribosome binding site
agaaaggaggtga

SEQ ID NO: 7 - DNA, 23 bp, Artificial Sequence
  Description of Artificial Sequenceribosome binding site
ttaagaaaggaggtgannnnatg

SEQ ID NO: 8 - DNA, 23 bp, Artificial Sequence
  Description of Artificial Sequenceribosome binding site
ttagaaaggaggtgannnnnatg

SEQ ID NO: 9 - DNA, 23 bp, Artificial Sequence
  Description of Artificial Sequenceribosome binding site
agaaaggaggtgannnnnnnatg

SEQ ID NO: 10 - DNA, 22 bp, Artificial Sequence
  Description of Artificial Sequenceribosome binding site
agaaaggaggtgannnnnnatg

SEQ ID NO: 11 - DNA, 25 bp, Artificial Sequence
  Description of Artificial Sequenceribosome binding site
ccctctagaaggaggagaaaacatg

SEQ ID NO: 12 - DNA, 24 bp, Artificial Sequence
  Description of Artificial Sequenceribosome binding site
ccctctagaggaggagaaaacatg

SEQ ID NO: 13 - DNA, 23 bp, Artificial Sequence
  Description of Artificial Sequenceribosome binding site
ttagaaaggaggatttaaatatg

SEQ ID NO: 14 - DNA, 23 bp, Artificial Sequence
  Description of Artificial Sequenceribosome binding site
ttagaaaggaggtttaattaatg

SEQ ID NO: 15 - DNA, 23 bp, Artificial Sequence
  Description of Artificial Sequenceribosome binding site
ttagaaaggaggtgatttaaatg

SEQ ID NO: 16 - DNA, 23 bp, Artificial Sequence
  Description of Artificial Sequenceribosome binding site
ttagaaaggaggtgtttaaaatg

SEQ ID NO: 17 - DNA, 28 bp, Artificial Sequence
  Description of Artificial Sequenceribosome binding site
attcgagaaaggaggtgaatataatatg

SEQ ID NO: 18 - DNA, 27 bp, Artificial Sequence
  Description of Artificial Sequenceribosome binding site
attcgagaaaggaggtgaataataatg

SEQ ID NO: 19 - DNA, 28 bp, Artificial Sequence
  Description of Artificial Sequenceribosome binding site
attcgtagaaaggaggtgaattaatatg

SEQ ID NO: 20 - DNA, 6886 bp, Artificial Sequence
  Description of Artificial Sequence vector
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctaacaggaggaattaaccatggatccgagctcgacagtatcaagcacttcacaatctgggagctgaaagcccgccttatgtagctcatacttgacaaatccaaggtcaaaatggatattgtgggcgacaaaataagcgccgtcaagcaattggaatacttcttcagcaactgcttcaaatggctgttcattctcgaccatttgattagagattccagtaagctgctcaataaaagcagggattgatttatttggattaatgtattttgaaaaccgctcagtaatttgtccattttcgattacaaccgctgcgatttgtatgattttatcgcctttcttcggcgaattccctgttgtctctacatctataacaacgaaccgttgcttattcattaaaatggacacctcaattcttgcatacgacaaaagtgtaacacgttttgtacggaaatggagcggcaaaaccgttttactctcaaaatcttaaaagaaaacccccgataaagggggcttttcttctacaaaattgtacgggctggttcgttccccagcatttgttcaattttgttttgatcattcagaacagccactttcggctcatggcttgccgcttcttgatcagacatcattttgtaggaaataataatgaccttatctccttcctgcacaaggcgtgcggctgcaccgtttaagcatatgacgccgcttccccgtttaccaggaataatatacgtttcaagacgtgctccattattattattcacaatttgtactttttcattaggaagcattcccacagcatcaatgagatcctctagagtcgacctgcaggcatgcaagcttccgtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcaatcttcatccattccaaggtaaatcccccttcgccgtttctgttaccattatacaccttttgaaccttaacgtaaacgttaagttttaaaaaacaataaaaaagacgagcagcatacagcacccgtctttcactttcctgtttaagctaaacttcccgccactgacagagactctttttgaaggctttcagaaagcactcgatacgcgatctggagctgtaatataaaaaccttcttcaactaacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaagaccacattaaaaaatgtggtcttttgtgtttttttaaaggatttgagcgtagcgaaaaatccttttctttcttatcttgataataagggtaactattgcatgataagctgtcaaacatgagaattcccgttttcttctgcaagccaaaaaaccttccgttacaacgagaaggattcttcactttctaaagttcggcgagtttcatccctctgtcccagtccttttttggatcaaggcagactgctgcaatgtctatctattttaataataggtgcagttcgcaggcgatactgcccaatggaagtataccaaaatcaacgggcttgtaccaacacattagcccaattcgatatcggcagaatagatttttttaatgccttcgttcgtttctaaaagcagaacgccttcatcatctatacctaacgccttaccgtaaaaggttccgtttaacgttctggctctcatattagtgccaataccgagcgcatagctttcccataaaagcttaatcggcgtaaatccgtgcgtcatataatcccggtaccgtttctcaaagcatagtaaaatatgctggatgacgccggcccgatcaattttttccccagcagcttggctgaggcttgtcgcgatgtccttcaattcatctggaaaatcattaggctgctggttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 21 - DNA, 7140 bp, Artificial Sequence
  Description of Artificial Sequence vector
tcggcggccgcttcgtcgaccgaaacagcagttataaggcatgaagctgtccggtttttgcaaaagtggctgtgactgtaaaaagaaatcgaaaaagaccgttttgtgtgaaaacggtctttttgtttccttttaaccaactgccataactcgaggcctacctagcttccaagaaagatatcctaacagcacaagagcggaaagatgttttgttctacatccagaacaacctctgctaaaattcctgaaaaattttgcaaaaagttgttgactttatctacaaggtgtggtataataatcttaacaacagcaggacgctctagattagaaaggaggatttaaatatgagacagattactgatatttcacagctgaaagaagccataaaacaataccattcagagggcaagtcaatcggatttgttccgacgatggggtttctgcatgaggggcatttaaccttagcagacaaagcaagacaagaaaacgacgccgttattatgagtatttttgtgaatcctgcacaattcggccctaatgaagattttgaagcatatccgcgcgatattgagcgggatgcagctcttgcagaaaacgccggagtcgatattctttttacgccagatgctcatgatatgtatcccggtgaaaagaatgtcacgattcatgtagaaagacgcacagacgtgttatgcgggcgctcaagagaaggacattttgacggggtcgcgatcgtactgacgaagcttttcaatctagtcaagccgactcgtgcctatttcggtttaaaagatgcgcagcaggtagctgttgttgatgggttaatcagcgacttcttcatggatattgaattggttcctgtcgatacggtcagagaggaagacggcttagccaaaagctctcgcaatgtatacttaacagctgaggaaagaaaagaagcgcctaagctgtatcgggcccttcaaacaagtgcggaacttgtccaagccggtgaaagagatcctgaagcggtgataaaagctgcaaaagatatcattgaaacgactagcggaaccatagactatgtagagctttattcctatccggaactcgagcctgtgaatgaaattgctggaaagatgattctcgctgttgcagttgctttttcaaaagcgcgtttaatagataatatcattattgatattcgtagaaaggaggtgaattaatatgtatcgtacgatgatgagcggcaaacttcacagggcaactgttacggaagcaaacctgaactatgtgggaagcattacaattgatgaagatctcattgatgctgtgggaatgcttcctaatgaaaaagtacaaattgtgaataataataatggagcacgtcttgaaacgtatattattcctggtaaacggggaagcggcgtcatatgcttaaacggtgcagccgcacgccttgtgcaggaaggagataaggtcattattatttcctacaaaatgatgtctgatcaagaagcggcaagccatgagccgaaagtggctgttctgaatgatcaaaacaaaattgaacaaatgctggggaacgaaccagcccgtacaattttgtaaaggatcctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttggtaccgagacgatcgtcctctttgttgtagcccatcacttttgctgaagagtaggagccgaaagtgacggcgtattcattgagcggcagctgagtcgcaccgacagaaatcgcttctcttgatgtgcccggcgatccgactgtccagccgttcggtccgctgttgccgtttgaggtaacagcgacaacgccttctgacatggcccagtcaagcgctgtgcttgtcgcccagtccgggttgtttaaagagtttccgagagacaggttcatcacatctgccccgtcctgcactgcacgttccacgcccgcgatgacgttttccgttgtgccgcttccgccaggccctaacacacgataagcaagaagtgtggcatcaggcgctacgcctttaatcgttccgtttgcagccacagttccggctacgtgtgtgccatggtcagttgcctcgcccctcggatcgccggttggtgtttcttttggatcgtaatcattgtccacaaaatcgtatcctttatattgtccaaagtttttcttcagatctgggtgattgtattcaaccccagtgtcaataatcgccaccttgatgccttttcctgtgtagcctaaatcccatgcatcgtttgctccgatataaggcgcactgtcatccatttgcggagatacggcgtcttcggagattgtggggaattctcatgtttgacagcttatcatgcaatagttacccttattatcaagataagaaagaaaaggatttttcgctacgctcaaatcctttaaaaaaacacaaaagaccacattttttaatgtggtctttattcttcaactaaagcacccattagttcaacaaacgaaaattggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttacagtcggttttctaatgtcactaacctgccccgttagttgaagaacgaagcggccgcaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcg

SEQ ID NO: 22 - DNA, 6725 bp, Artificial Sequence
  Description of Artificial Sequence vector
tcggcggccgcttcgtcgaccgaaacagcagttataaggcatgaagctgtccggtttttgcaaaagtggctgtgactgtaaaaagaaatcgaaaaagaccgttttgtgtgaaaacggtctttttgtttccttttaaccaactgccataactcgaggcctacctagcttccaagaaagatatcctaacagcacaagagcggaaagatgttttgttctacatccagaacaacctctgctaaaattcctgaaaaattttgcaaaaagttgttgactttatctacaaggtgtggtataataatcttaacaacagcaggacgctctagaaggaggagaaaacatgaaaacaaaactggattttctaaaaatgaaggagtctgaagaaccgattgtcatgctgaccgcttatgattatccggcagctaaacttgctgaacaagcgggagttgacatgattttagtcggtgattcacttggaatggtcgtcctcggccttgattcaactgtcggtgtgacagttgcggacatgatccatcatacaaaagccgttaaaaggggtgcgccgaatacctttattgtgacagatatgccgtttatgtcttatcacctgtctaaggaagatacgctgaaaaatgcagcggctatcgttcaggaaagcggagctgacgcactgaagcttgagggcggagaaggcgtgtttgaatccattcgcgcattgacgcttggaggcattccagtagtcagtcacttaggtttgacaccgcagtcagtcggcgtactgggcggctataaagtacagggcaaagacgaacaaagcgccaaaaaattaatagaagacagtataaaatgcgaagaagcaggagctatgatgcttgtgctggaatgtgtgccggcagaactcacagccaaaattgccgagacgctaagcataccggtcattggaatcggggctggtgtgaaagcggacggacaagttctcgtttatcatgatattatcggccacggtgttgagagaacacctaaatttgtaaagcaatatacgcgcattgatgaaaccatcgaaacagcaatcagcggatatgttcaggatgtaagacatcgtgctttccctgaacaaaagcattcctttcaaatgaaccagacagtgcttgacggcttgtacgggggaaaataagggggggatcctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttggtaccgagacgatcgtcctctttgttgtagcccatcacttttgctgaagagtaggagccgaaagtgacggcgtattcattgagcggcagctgagtcgcaccgacagaaatcgcttctcttgatgtgcccggcgatccgactgtccagccgttcggtccgctgttgccgtttgaggtaacagcgacaacgccttctgacatggcccagtcaagcgctgtgcttgtcgcccagtccgggttgtttaaagagtttccgagagacaggttcatcacatctgccccgtcctgcactgcacgttccacgcccgcgatgacgttttccgttgtgccgcttccgccaggccctaacacacgataagcaagaagtgtggcatcaggcgctacgcctttaatcgttccgtttgcagccacagttccggctacgtgtgtgccatggtcagttgcctcgcccctcggatcgccggttggtgtttcttttggatcgtaatcattgtccacaaaatcgtatcctttatattgtccaaagtttttcttcagatctgggtgattgtattcaaccccagtgtcaataatcgccaccttgatgccttttcctgtgtagcctaaatcccatgcatcgtttgctccgatataaggcgcactgtcatccatttgcggagatacggcgtcttcggagattgtggggaattctcatgtttgacagcttatcatgcaatagttacccttattatcaagataagaaagaaaaggatttttcgctacgctcaaatcctttaaaaaaacacaaaagaccacattttttaatgtggtctttattcttcaactaaagcacccattagttcaacaaacgaaaattggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttacagtcggttttctaatgtcactaacctgccccgttagttgaagaacgaagcggccgcaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcg

SEQ ID NO: 23 - DNA, 6806 bp, Artificial Sequence
  Description of Artificial Sequence vector
tcggcggccgcttcgtcgaccgaaacagcagttataaggcatgaagctgtccggtttttgcaaaagtggctgtgactgtaaaaagaaatcgaaaaagaccgttttgtgtgaaaacggtctttttgtttccttttaaccaactgccataactcgaggcctacctagcttccaagaaagatatcctaacagcacaagagcggaaagatgttttgttctacatccagaacaacctctgctaaaattcctgaaaaattttgcaaaaagttgttgactttatctacaaggtgtggtataataatcttaacaacagcaggacgctctagacaattgagatcttaagaaaggaggtgttaattaatgaagattggaatcattggcggaggctccgttggtcttttatgcgcctattatttgtcactttatcacgacgtgactgttgtgacgaggcggcaagaacaggctgcggccattcagtctgaaggaatccggctttataaaggcggggaggaattcagggctgattgcagtgcggacacgagtatcaattcggactttgacctgcttgtcgtgacagtgaagcagcatcagcttcaatctgttttttcgtcgcttgaacgaatcgggaagacgaatatattatttttgcaaaacggcatggggcatatccacgacctaaaagactggcacgttggccattccatttatgttggaatcgttgagcacggagctgtaagaaaatcggatacagctgttgatcatacaggcctaggtgcgataaaatggagcgcgttcgacgatgctgaaccagaccggctgaacatcttgtttcagcataaccattcggattttccgatttattatgagacggattggtaccgtctgctgacgggcaagctgattgtaaatgcgtgtattaatcctttaactgcgttattgcaagtgaaaaatggagaactgctgacaacgccagcttatctggcttttatgaagctggtatttcaggaggcatgccgcattttaaaacttgaaaatgaagaaaaggcttgggagcgggttcaggccgtttgtgggcaaacgaaagagaatcgttcatcaatgctggttgacgtcattggaggccggcagacggaagctgacgccattatcggatacttattgaaggaagcaagtcttcaaggtcttgatgccgtccacctagagtttttatatggcagcatcaaagcattggagcgaaataccaacaaagtggtttactaaggatcctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttggtaccgagacgatcgtcctctttgttgtagcccatcacttttgctgaagagtaggagccgaaagtgacggcgtattcattgagcggcagctgagtcgcaccgacagaaatcgcttctcttgatgtgcccggcgatccgactgtccagccgttcggtccgctgttgccgtttgaggtaacagcgacaacgccttctgacatggcccagtcaagcgctgtgcttgtcgcccagtccgggttgtttaaagagtttccgagagacaggttcatcacatctgccccgtcctgcactgcacgttccacgcccgcgatgacgttttccgttgtgccgcttccgccaggccctaacacacgataagcaagaagtgtggcatcaggcgctacgcctttaatcgttccgtttgcagccacagttccggctacgtgtgtgccatggtcagttgcctcgcccctcggatcgccggttggtgtttcttttggatcgtaatcattgtccacaaaatcgtatcctttatattgtccaaagtttttcttcagatctgggtgattgtattcaaccccagtgtcaataatcgccaccttgatgccttttcctgtgtagcctaaatcccatgcatcgtttgctccgatataaggcgcactgtcatccatttgcggagatacggcgtcttcggagattgtggggaattctcatgtttgacagcttatcatgcaatagttacccttattatcaagataagaaagaaaaggatttttcgctacgctcaaatcctttaaaaaaacacaaaagaccacattttttaatgtggtctttattcttcaactaaagcacccattagttcaacaaacgaaaattggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttacagtcggttttctaatgtcactaacctgccccgttagttgaagaacgaagcggccgcaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcg

SEQ ID NO: 24 - DNA, 3867 bp, Artificial Sequence
  Description of Artificial Sequence vector
aagctttctcaagaagcgaacaagaaaaaagaagagcagattaaacagcttcaagagtttgtcgctagattcagcgccaatgcgtctaaatctaagcaggctacatcaagaaagaaacttctcgaaaaaatcacgctggatgatattaaaccgtcttcccgccgctatccttatgttaacttcacgccggaacgggaaatcggaaatgatgttcttcgcgtggaaggcttaacaaaaacaatcgatggcgtaaaggtgcttgacaatgtcagctttattatgaatcgagaagataaaattgctttcactggccgaaatgaacttgctgttactacgctgtttaaaatcatttccggggaaatggaagctgacagcggaacgtttaaatggggtgttaccacatctcaagcgtattttccaaaagacaacagcgaatatttcgaaggcagtgatctgaaccttgtagactggcttcgccaatattctccgcacgaccaaagtgagagctttttacgcggtttcttaggacgcatgctgttctctggagaagaagtccacaaaaaagcaaatgtactttccgggggagaaaaggtccgctgtatgctgtcgaaagcgatgctttctggcgccaatattttaattttggatgagccgaccaaccatttagacctagagtccattacagcgctcaataacggcttaatcagctttaaaggcgctatgctgtttacttcccatgaccatcagtttgtgcagaccattgccaacagaattatagaaattacacctaacggcatcgtcgataagcaaatgagctatgacgagttccttgaaaatgctgatgtgcagaaaaaattgactgaactatacgccgaataaaaaagcagagatttctctgctttttttgatacctaaatgtgaaaggagatcacaacatgaaatttttggttgtcggagcaggtggagtaggcgggtatattggcggacggctttcggagaaaggaaatgatgtgacatttctcgtgcgccaaaaacgagctgagcagcttaaaaaaaccgggcttgtcatccatagtgaaaaagggaatgtatcatttcagcccgaactaatcagtgccggagaaacagggcaatttgatgtcgttatcattgcttctaaagcatactcgcttggtcaagtgatagaggatgtcaaaccatttatccatcaagaatctgtcattatcccttttttaaatgggtaccgccactatgagcagctatttgcggcattttcaaaagaacaggtgctgggcggcctgtgttttatagaaagtgctttagacaacaaaggagaaattcatcatacgagcgcatcgcatcgttttgtatttggagaatggaacggcgagcgtacggagcggataagagcgcttgaagaggcattttcaggtgtgaaggctgaagtcatcattagcgggcatatcgagaagatcccctgcagcaatagttacccttattatcaagataagaaagaaaaggatttttcgctacgctcaaatcctttaaaaaaacacaaaagaccacattttttaatgtggtctttattcttcaactaaagcacccattagttcaacaaacgaaaattggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttacagtcggttttctaatgtcactaacctgccccgttagttgaagaaggtttttatattacagctcccgggagatctgggatcactagtccaaacgacagaaggcgaccacctgcatggatttttgattgaaaaagcaaaacgtttatctctcgctgcaccagtattagaaaccgtttatgcgaatctgcaaatgtatgaagcagaaaaataaaaaaaggaggcggaaaagcctccttttatttacttaaaaagcccaatttccgtttctgaagataggctctcttttcccgtctgccgtaattccgtcaatattcatatccttagaaccgatcataaagtccacgtgtgtaatgctttcatttaggccttctttgacaagctcttcacgagacatctgctttccgccttcaatattaaaggcataggcgcttccgatcgccaaatgatttgacgcgttttcatcaaacagcgtgttatagaaaagaatgtttgattgtgatataggcgaatcgtaaggaacaagtgccacttcacctaaatagtgagaaccttcatctgtttccaccagttcttttaaaatatcctcacctttttcagctttaatgtcgactatacggccattttcaaacgtcagggtgaaattttcaataatatttccgccgtagcttaatggttttgtgcttgataccactccgtcaaccccgtctttttgcggcagcgtgaacacttcttctgtcggcatattggccataaactcatggccactttcattcacgcttcccgcacctgcccaaacatgcttcctaggcagcttaattgttagatcagttccttctgcttgataatgtaaggcagcgtaatgtctctcgttcaaatggtcaactttttcatgaagattttggtcatgattgatccacgcctgaaccgggttgtcttcatttacgcgcgtcgctttaaaaatttcttcccacagaaggtggatcgcttcctcctctgatttgccaggaaacaccttgtgagcccagcctgctgatgccgcacctacgacagtccagctgactttgtctgattgaatatattgtctgtatgtatgtaatgctttgcctgctgctttttggaatgccgcaatccgtttggaatctataccttttagcaagtctgggttcgacgacacaacagaaatgaaagcagctccatttttggcaagctcttctctgccttttgcttcccattcaggatattcttcaaatgcttcaaacggcgcaagttcgtattttaatttggcgacttcgtcatcctgccaattcacggtgacgttttttgcgcccttttcatatgcgtgttttacaattaaacggacaaaatcccgaacgtctgttgaagcatttacgactacatactggcctttttggacattaacgc

SEQ ID NO: 25 - DNA, 8704 bp, Artificial Sequence
  Description of Artificial Sequence vector
gcggccgcttcgtcgaccgaaacagcagttataaggcatgaagctgtccggtttttgcaaaagtggctgtgactgtaaaaagaaatcgaaaaagaccgttttgtgtgaaaacggtctttttgtttccttttaaccaactgccataactcgaggcctacctagcttccaagaaagatatcctaacagcacaagagcggaaagatgttttgttctacatccagaacaacctctgctaaaattcctgaaaaattttgcaaaaagttgttgactttatctacaaggtgtggtataataatcttaacaacagcaggacgctctagaggaggagactaacatgaaatttttggttgtcggagcaggtggagtaggcgggtatattggcggacggctttcggagaaaggaaatgatgtgacatttctcgtgcgccaaaaacgagctgagcagcttaaaaaaaccgggcttgtcatccatagtgaaaaagggaatgtatcatttcagcccgaactaatcagtgccggagaaacagggcaatttgatgtcgttatcattgcttctaaagcatactcgcttggtcaagtgatagaggatgtcaaaccatttatccatcaagaatctgtcattatcccttttttaaatgggtaccgccactatgagcagctatttgcggcattttcaaaagaacaggtgctgggcggcctgtgttttatagaaagtgctttagacaacaaaggagaaattcatcatacgagcgcatcgcatcgttttgtatttggagaatggaacggcgagcgtacggagcggataagagcgcttgaagaggcattttcaggtgtgaaggctgaagtcatcattagcgggcatatcgagaaggacatttggaaaaagtatctctttattgcagcgcaagcggggatcacaacgttatttcaacgaccgcttggcccaatcctcgccacagaagccggacgtcacacggcccaaactcttattggggaaatttgcactgttttacgaaaagaaggtgttccggctgatccggctcttgaggaagagagctttcgtacgatgaccagcatgtcttaccatatgaagtcctccatgcttcgggatatggaaaacggccaaacgacagaaggcgaccacctgcatggatttttgattgaaaaagcaaaacgtttatctctcgctgcaccagtattagaaaccgtttatgcgaatctgcaaatgtatgaagcagaaaaataaaaaaaggaggcggaaaagcctccttttatttacttaaaaagcccaatttccgtttctgaagataggctctcttttcccgtctgccgggatcctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttggtacccagaaaaagcggcaaaagcggctgttaaaaaagcgaaatcgaagaagctgtctgccgctaagacggaatatcaaaagcgttctgctgttgtgtcatctttaaaagtcacagccgatgaatcccagcaagatgtcctaaaatacttgaacacccagaaagataaaggaaatgcagaccaaattcattcttattatgtggtgaacgggattgctgttcatgcctcaaaagaggttatggaaaaagtggtgcagtttcccgaagtggaaaaggtgcttcctaatgagaaacggcagctttttaagtcatcctccccatttaatatgaaaaaagcacagaaagctattaaagcaactgacggtgtggaatggaatgtagaccaaatcgatgccccaaaagcttgggcacttggatatgatggaactggcacggttgttgcgtccattgataccggggtggaatggaatcatccggcattaaaagagaaatatcgcggatataatccggaaaatcctaatgagcctgaaaatgaaatgaactggtatgatgccgtagcaggcgaggcaagcccttatgatgatttggctcatggaacccacgtgacaggcacgatggtgggctctgaacctgatggaacaaatcaaatcggtgtagcacctggcgcaaaatggattgctgttaaagcgttctctgaagatggcggcactgatgctgacattttggaagctggtgaatgggttttagcaccaaaggacgcggaaggaaatccccacccggaaatggctcctgatgttgtcaataactcatggggagggggctctggacttgatgaatggtacagagacatggtcaatgcctggcgttcggccgatattttccctgagttttcagcggggaatacggatctctttattcccggcgggcctggttctatcgcaaatccggcaaactatccagaatcgtttgcaactggagcgactgagaattccaattccccatggagagaaaagaaaatcgctaatgttgattactttgaacttctgcatattcttgaatttaaaaaggctgaaagagtaaaagattgtgctgaaatattagagtataaacaaaatcgtgaaacaggcgaaagaaagttgtatcgagtgtggttttgtaaatccaggctttgtccaatgtgcaactggaggagagcaatgaaacatggcattcagtcacaaaaggttgttgctgaagttattaaacaaaagccaacagttcgttggttgtttctcacattaacagttaaaaatgtttatgatggcgaagaattaaataagagtttgtcagatatggctcaaggatttcgccgaatgatgcaatataaaaaaattaataaaaatcttgttggttttatgcgtgcaacggaagtgacaataaataataaagataattcttataatcagcacatgcatgtattggtatgtgtggaaccaacttattttaagaatacagaaaactacgtgaatcaaaaacaatggattcaattttggaaaaaggcaatgaaattagactatgatccaaatgtaaaagttcaaatgattcgaccgaaaaataaatataaatcggatatacaatcggcaattgacgaaactgcaaaatatcctgtaaaggatacggattttatgaccgatgatgaagaaaagaatttgaaacgtttgtctgatttggaggaaggtttacaccgtaaaaggttaatctcctatggtggtttgttaaaagaaatacataaaaaattaaaccttgatgacacagaagaaggcgatttgattcatacagatgatgacgaaaaagccgatgaagatggattttctattattgcaatgtggaattgggaacggaaaaattattttattaaagagtagttcaacaaacgggccatattgttgtataagtgatgaaatactgaatttaaaacttagtttatatgtggtaaaatgttttaatcaagtttaggaggaattaattatgaagtgtaatgaatgtaacagggttcaattaaaagagggaagcgtatcattaaccctataaactacgtctgccctcattattggagggtgaaatgtgaatacatcctattcacaatcgaatttacgacacaaccaaattttaatttggctttgcattttatctttttttagcgtattaaatgaaatggttttgaacgtctcattacctgatattgcaaatgattttaataaaccacctgcgagtacaaactgggtgaacacagcctttatgttaaccttttccattggaacagctgtatatggaaagctatctgatcaattaggcatcaaaaggttactcctatttggaattataataaattgtttcgggtcggtaattgggtttgttggccattctttcttttccttacttattatggctcgttttattcaaggggctggtgcagctgcatttccagcactcgtaatggttgtagttgcgcgctatattccaaaggaaaataggggtaaagcatttggtcttattggatcgatagtagccatgggagaaggagtcggtccagcgattggtggaatgatagcccattatattcattggtcctatcttctactcattcctatgataacaattatcactgttccgtttcttatgaaattattaaagaaagaagtaaggataaaaggtcattttgatatcaaaggaattatactaatgtctgtaggcattgtattttttatgttgtttacaacatcatatagcatttcttttcttatcgttagcgtgctgtcattcctgatatttgtaaaacatatcaggaaagtaacagatccttttgttgatcccggattagggaaaaatataccttttatgattggagttctttgtgggggaattatatttggaacagtagcagggtttgtctctatggttccttatatgatgaaagatgttcaccagctaagtactgccgaaatcggaagtgtaattattttccctggaacaatgagtgtcattattttcggctacattggtgggatacttgttgatagaagaggtcctttatacgtgttaaacatcggagttacatttctttctgttagctttttaactgcttcctttcttttagaaacaacatcatggttcatgacaattataatcgtatttgttttaggtgggctttcgttcaccaaaacagttatatcaacaattgtttcaagtagcttgaaacagcaggaagctggtgctggaatgagtttgcttaactttaccagctttttatcagagggaacaggtattgcaattgtaggtggtttattatccatacccttacttgatcaaaggttgttacctatggaagttgatcagtcaacttatctgtatagtaatttgttattacttttttcaggaatcattgtcattagttggctggttaccttgaatgtatataaacattctcaaagggatttctaaatcgttaagggatcaactttgggagagagttcaaaattgatcctttttttataacagttcgaagcggccgcaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcg

(19) United States
(12) Patent Application Publication
(10) Pub. No.: US 2004/0048343 A1
(43) Pub. Date: Mar. 11, 2004

(54) Method and microorganisms for the production of 3-(2-hydroxy-3-methyl-butyrylamino)-propionic acid (hmbpa)

(76) Inventors: Theron Hermann, Kinnelon, NJ (US); Thomas A. Patterson, Attleboro, MA (US); Janice G. Pero, Lexington, MA (US); R. Rogers Yocum, Lexington, MA (US); Kai-Uwe Baldenius, Ludwigshafen (DE); Christine Beck, Mannheim (DE)

Correspondence Address: Lahive & Cockfield, Counsellors at Law, 28 State Street, Boston, MA, 02109-1784, US

(21) Appl. No.: 10/466,642

(22) PCT Filed: Jan. 19, 2002
(86) PCT No.: PCT/US02/01887

(51) Int. Cl. C12P 13/04; C12N 1/21

(52) U.S. Cl. 435/106; 435/252.3

(57) ABSTRACT

The present invention features methods of producting 3-(2-hydroxy-3-methyl-butyrylamino)-propionic acid (“HMBPA”) and α-hydroxyisovalerate (“α-HIV”) utilizing microorganisms having modified pantothenate biosynthetic enzyme activities. Recombinant microorganisms and conditions for culturing same are also featured. Also featured are compositions including HMBPA and compositions including α-HIV.

### RELATED APPLICATIONS

The present invention claims the benefit of prior-filed provisional Patent Application Serial No. 60/263,053, filed Jan. 19, 2001 (pending). The present invention is also related to U.S. patent application Ser. No. 09/667,569, filed Sep. 21, 2000 (pending), which is a continuation-in-part of U.S. patent application Ser. No. 09/400,494, filed Sep. 21, 1999 (abandoned). U.S. patent application Ser. No. 09/667,569 also claims the benefit of prior-filed provisional Patent Application Serial No. 60/210,072, filed Jun. 7, 2000, provisional Patent Application Serial No. 60/221,836, filed Jul. 28, 2000, and provisional Patent Application Serial No. 60/227,860, filed Aug. 24, 2000. The entire content of each of the above-referenced applications is incorporated herein by this reference.

### BACKGROUND OF THE INVENTION

Conventional means of synthesizing chemical compounds is via synthesis from bulk chemicals, a process which is limited by factors such as substrate availability and/or cost, difficulty in resolving complex mixtures of products, complexities in synthesizing large quantities of compounds in purified form, and difficulty in producing chiral compounds. Accordingly, researchers have recently looked to bacterial or microbial systems that express enzymes useful for various biosynthetic processes, for example, in the synthesis of pharmaceutical compounds, research reagents, nutriceuticals, vitamins, nutritional supplements, antibiotic compounds and the like. In particular, bioconversion processes have been evaluated as a means of favoring production of preferred compounds and recently methods of direct microbial synthesis have been the focus of much research in the areas of pharmaceuticals and agriculture.

### SUMMARY OF THE INVENTION

The present invention relates to a processes for the direct microbial synthesis of [R]-3-(2-hydroxy-3-methyl-butyrylamino)-propionic acid or 3-(2-hydroxy-3-methyl-butyrylamino)-propionic acid (“HMBPA”), referred to interchangeably herein as “β-alanine 2-(R)-hydroxyisolvalerate”, “β-alanine 2-hydroxyisolvalerate”, “β-alanyl-α-hydroxyisovalarate”, N-(2-hydroxy-3-methyl-1-oxobutyl)-β-alanine (“HMOBA”) and/or “fantothenate”. In particular, it has been discovered that in microorganisms engineered to overexpress certain enzymes conventionally associated with pantothenate and/or isoleucine-valine (ilv) biosynthesis, an alternative biosynthetic pathway is present that competes for key precursors of pantothenate biosynthesis, namely α-ketoisovalerate (α-KIV) and β-alanine. α-KIV is converted to α-hydroxyisovalerate (α-HIV) catalyzed by various reductase enzymes and α-HIV is subsequently condensed with β-alanine to produce HMBPA.

In one embodiment, the invention features a process for the production of 3-(2-hydroxy-3-methyl-butyrylamino)-propionic acid (HMBPA) that includes culturing a microorganism having increased keto reductase activity or increased pantothenate synthetase activity in the presence of excess α-ketoisovalerate and excess β-alanine, such that HMBPA is produced. In another embodiment, the invention features a process for the production of 3-(2-hydroxy-3-methyl-butyrylamino)-propionic acid (HMBPA) that includes culturing a microorganism having increased keto reductase activity and increased pantothenate synthetase activity in the presence of excess α-ketoisovalerate and excess β-alanine, such that HMBPA is produced. In one embodiment, the microorganism has a modified panE gene, for example, a modified panel gene and/or a modified panE2 gene (e.g., the panE gene is overexpressed, deregulated or present in multiple copies). In another embodiment, the microorganism has a modified panC gene (e.g., the panC gene is overexpressed, deregulated or present in multiple copies). In another embodiment, the microorganism further has increased acetohydroxyacid isomeroreductase activity. In another embodiment, the microorganism is cultured under conditions of increased acetohydroxyacid isomeroreductase activity in the presence of excess α-ketoisovalerate and excess β-alanine, such that HMBPA is produced. In yet another embodiment, the microorganism comprises a modified ilvC gene (e.g., the ilvC gene is overexpressed, deregulated or present in multiple copies). In yet another embodiment, the microorganism further has reduced ketopantoate hydroxymethyltransferase activity (e.g., has a modified panB gene, for example a panB gene that has been deleted.

In another aspect, the invention features a process for the production of 3-(2-hydroxy-3-methyl-butyrylamino)-propionic acid (HMBPA) that includes culturing a microorganism having reduced ketopantoate hydroxymethyltransferase activity in the presence of excess α-ketoisovalerate and excess β-alanine, such that HMBPA is produced. In another aspect, the invention features a method for enhancing production of 3-(2-hydroxy-3-methyl-butyrylamino)-propionic acid (HMBPA) relative to pantothenate that includes culturing a recombinant microorganism under conditions such that the HMBPA production is enhanced relative to pantothenate production. In another aspect, the invention features a process for the production of 2-hydroxyisovaleric acid (α-HIV) that includes culturing a microorganism which overexpresses PanE1 or PanE2 and which further has reduced PanC or PanD activity under conditions such that α-HIV is produced. In another aspect, the invention features a process for the production of 3-(2-hydroxy-3-methyl-butyrylamino)-propionic acid (HMBPA) that includes culturing a recombinant microorganism having decreased expression or activity of serA or glyA under conditions such that HMBPA is produced. In another aspect, the invention features a process for the production of 3-(2-hydroxy-3-methyl-butyrylamino)-propionic acid (HMBPA) that includes culturing a recombinant microorganism having decreased expression or activity of serA and glyA under conditions such that HMBPA is produced. Conditions for culturing the above described microorganisms include, for example, conditions of increased steady state glucose, conditions of decreased steady state dissolved oxygen, and/or cultured under conditions of decreased serine. Products produced according to the above described processes and/or methods are also featured. Also featured are recombinant microorganisms utilized in the above-described methods.

Compounds produced according to the methodologies of the present invention have a variety of uses. For example, HMBPA can be used to synthesize inhibitors of HMG CoA Reductase (II) (Gordon et al. Bio. Med. Chem. Lett. 1(3):161 (1991). Inhibitors of HMG CoA Reductase (II) have been studied for use as in the treatment of hypercholesterolaemia and coronary atherosclerosis progression. Inhibitors of HMG CoA Reductase also have been used to reduce risk of cardiovascular events in patients at risk. Moreover, the HMBPA precursor 2-hydroxyisovalerate (α-HIV) has been demonstrated to have nutriceutical properties, for example, in the prevention of aging of the skin. In particular, α-hydroxy acids, such as α-HIV (or 2-hydroxyvaline), can be used to synthesize α-hydroxy esters which have been found to induce increased skin thickness by increasing biosyntheses of glycosaminoglycans, proteoglycans, collagen, elastin, and other dermal components. The compounds can be used to treat skin disorders such as age spots, skin lines, wrinkles, photoaging and aging.

Other features and advantages of the invention will be apparent from the following detailed description and claims.

### BRIEF DESCRIPTION OF THE DRAWINGS

FIG. 1 is a schematic representation of the pantothenate and isoleucine-valine (ilv) biosynthetic pathways. Pantothenate biosynthetic enzymes are depicted in bold and their corresponding genes indicated in italics. Isoleucine-valine (ilv) biosynthetic enzymes are depicted in bold italics and their corresponding genes indicated in italics.

FIG. 2 is a schematic representation of the biosynthetic pathway leading to [R]-3-(2-hydroxy-3-methyl-butyrylamino)-propionic acid (“HMBPA”) in B. subtilis.

FIG. 3 is a schematic depiction of the structure of [R]-3-(2-hydroxy-3methyl-butyrylamino)-propionic acid (“HMBPA”).

FIG. 4 is a HPLC chromatogram of a sample of medium from a 14 L fermentation of PA824.

FIG. 5 is a mass spectrum depicting the relative monoisotopic mass of [R]-3-(2-hydroxy-3-methyl-butyrylamino)-propionic acid.

FIG. 6 depicts an alignment of the C-terminal amino acids from known or suspected PanB proteins.

FIG. 7 is a schematic representation of the construction of the plasmid pAN624.

FIG. 8 is a schematic representation of the construction of the plasmid pAN620.

FIG. 9 is a schematic representation of the construction of the plasmid pAN636.

FIG. 10 is a schematic representation of the construction of the plasmid pAN637 which allows selection for single or multiple copies using chloramphenicol.

FIG. 11 is a schematic representation of the construction of the plasmid pAN238, a plasmid for overexpressing B. subtilis panE2 from the P₂₆ promoter.

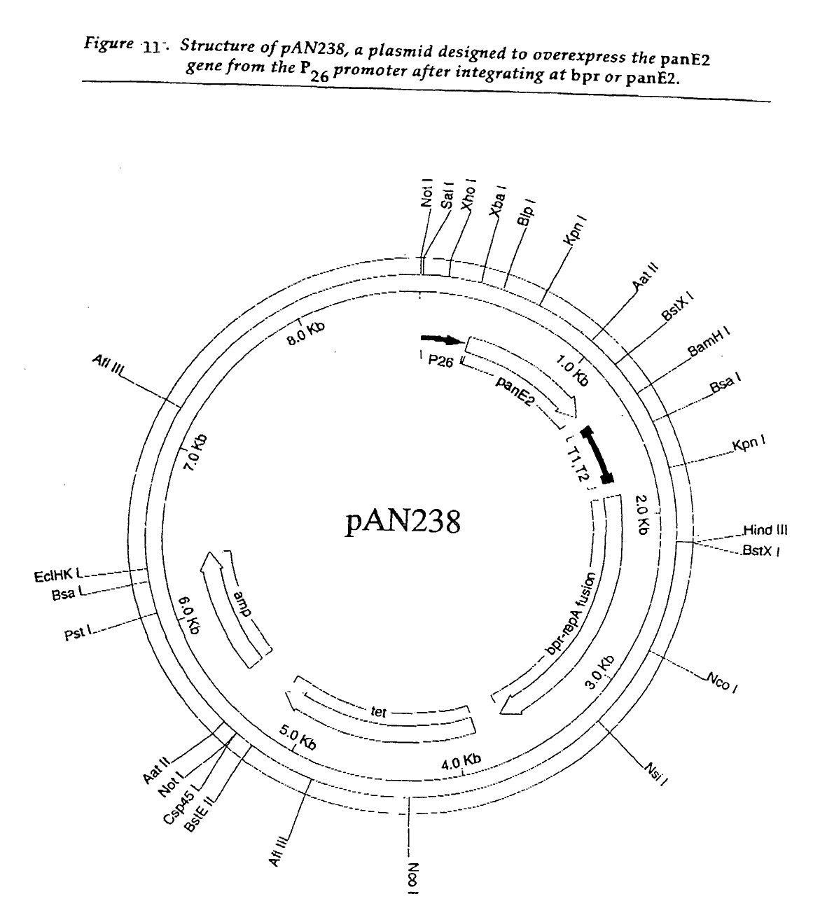

### DETAILED DESCRIPTION OF THE INVENTION

The present invention is based, at least in part, on the discovery of a novel biosynthetic pathway in bacteria, namely the [R]-3-(2-hydroxy-3-methyl-butyrylamino)propionic acid (“HMBPA”) biosynthetic pathway. In particular, it has been discovered that bacteria are capable of generating HMBPA from α-ketoisovalerate (α-KIV), a key product of the isoleucine-valine (ilv) biosynthetic pathway and precursor of the pantothenate biosynthetic pathway. Production of HMBPA in bacteria involves at least the pantothenate biosynthetic enzymes ketopantoate reductase (the panE1 gene product) and/or acetohydroxyacid isomeroreductase (the ilvC gene product) and results from the condensation of 2-hydroxyisovaleric acid (α-HIV), formed by reduction of α-KIV, and β-alanine, the latter reaction being catalyzed by the pantothenate biosynthetic enzyme pantothenate synthetase (the panC gene product). Production of HMBPA is achieved by increasing ketopantoate reductase (e.g., PanE1) and/or PanE2 and/or acetohydroxyacid isomeroreductase activities (e.g., IlvC) in microorganisms, for example, by overexpressing or deregulating the genes encoding said enzymes. Optimal production of HMBPA is achieved by decreasing or deleting ketopantoate hydroxymethyltransferase activity (the panB gene product) in microorganisms, for example, by modifying or deleting the panB gene which encodes ketopantoate hydroxymethyltransferase (e.g., PanB), optionally in addition to increasing ketopantoate reductase and/or PanE2 and/or acetohydroxyacid isomeroreductase activities in said microorganisms. The substrates α-KIV and β-alanine are required for HMBPA production, the latter provided, for example, by β-alanine feeding and/or increased aspartate-α-decarboxylate activity (the panD gene product). Increasing substrate concentration (i.e., α-KIV and/or β-alanine) further enhances production of HMBPA. α-KIV production can be increased by overexpressing ilvBNCD genes and/or alsS. HMBPA production can further be increased by limiting serine availability or synthesis in appropriately engineered microorganisms.

In order that the present invention may be more readily understood, certain terms are first defined herein.

The term “pantothenate biosynthetic pathway” includes the biosynthetic pathway involving pantothenate biosynthetic enzymes (e.g., polypeptides encoded by biosynthetic enzyme-encoding genes), compounds (e.g., precursors, substrates, intermediates or products), cofactors and the like utilized in the formation or synthesis of pantothenate. The term “pantothenate biosynthetic pathway” includes the biosynthetic pathway leading to the synthesis of pantothenate in microorganisms (e.g., in vivo) as well as the biosynthetic pathway leading to the synthesis of pantothenate in vitro.

The term “pantothenate biosynthetic enzyme” includes any enzyme utilized in the formation of a compound (e.g., intermediate or product) of the pantothenate biosynthetic pathway. For example, synthesis of pantoate from α-ketoisovalerate (α-KIV) proceeds via the intermediate, ketopantoate. Formation of ketopantoate is catalyzed by the pantothenate biosynthetic enzyme ketopantoate hydroxymethyltransferase (the panB gene product). Formation of pantoate is catalyzed by the pantothenate biosynthetic enzyme ketopantoate reductase (the panE gene product). Synthesis of β-alanine from aspartate is catalyzed by the pantothenate biosynthetic enzyme aspartate-α-decarboxylase (the panD gene product). Formation of pantothenate from pantoate and β-alanine (e.g., condensation) is catalyzed by the pantothenate biosynthetic enzyme pantothenate synthetase (the panC gene product). Based on the newly discovered HMBPA biosynthesis pathway, pantothenate biosynthetic enzymes may also perform an alternative function as enzymes in the HMBPA biosynthetic pathway described herein.

The term “pantothenate” includes the free acid form of pantothenate, also referred to as “pantothenic acid” as well as any salt thereof (e.g., derived by replacing the acidic hydrogen of pantothenate or pantothenic acid with a cation, for example, calcium, sodium, potassium, ammonium), also referred to as a “pantothenate salt”. The term “pantothenate” also includes alcohol derivatives of pantothenate. Preferred pantothenate salts are calcium pantothenate or sodium pantothenate. A preferred alcohol derivative is pantothenol. Pantothenate salts and/or alcohols of the present invention include salts and/or alcohols prepared via conventional methods from the free acids described herein. In another embodiment, calcium pantothenate is synthesized directly by a microorganism of the present invention. A pantothenate salt of the present invention can likewise be converted to a free acid form of pantothenate or pantothenic acid by conventional methodology.

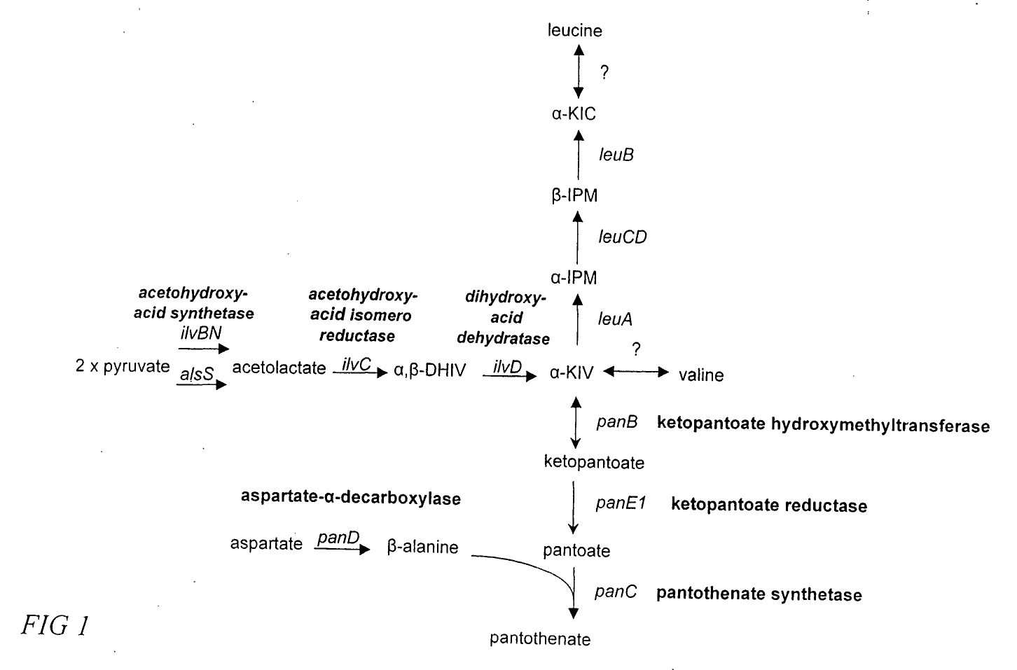

The term “isoleucine-valine biosynthetic pathway” includes the biosynthetic pathway involving isoleucine-valine biosynthetic enzymes (e.g., polypeptides encoded by biosynthetic enzyme-encoding genes), compounds (e.g., precursors, substrates, intermediates or products), cofactors and the like utilized in the formation or synthesis of conversion of pyruvate to valine or isoleucine. The term “isoleucine-valine biosynthetic pathway” includes the biosynthetic pathway leading to the synthesis of valine or isoleucine in microorganisms (e.g., in vivo) as well as the biosynthetic pathway leading to the synthesis of valine or isoleucine in vitro. FIG. 1 includes a schematic representation of the isoleucine-valine biosynthetic pathway. Isoleucine-valine biosynthetic enzymes are depicted in bold italics and their corresponding genes indicated in italics

The term “isoleucine-valine biosynthetic enzyme” includes any enzyme utilized in the formation of a compound (e.g., intermediate or product) of the isoleucine-valine biosynthetic pathway. According to FIG. 1, synthesis of valine from pyruvate proceeds via the intermediates, acetolactate, α,β-dihydroxyisovalerate (α,β-DHIV) and α-ketoisovalerate (α-KIV). Formation of acetolactate from pyruvate is catalyzed by the isoleucine-valine biosynthetic enzyme acetohydroxyacid synthetase (the ilvBN gene product, or alternatively, the alsS gene product). Formation of α,β-DHIV from acetolactate is catalyzed by the isoleucine-valine biosynthetic enzyme acetohydroxyacidisomero reductase (the ilvC gene product). Synthesis of α-KIV from α,β-DHIV is catalyzed by the isoleucine-valine biosynthetic enzyme dihydroxyacid dehydratase (the ilvD gene product). Moreover, valine and isoleucine can be interconverted with their respective α-keto compounds by branched chain amino acid transaminases. Based on the newly discovered HMBPA biosynthesis pathway, isoleucine-valine biosynthetic enzymes may also perform an alternative function as enzymes in the HMBPA biosynthetic pathway described herein.

The term “3-(2-hydroxy-3-methyl-butyrylamino)-propionic acid (“HMBPA”) biosynthetic pathway” includes the alternative biosynthetic pathway involving biosynthetic enzymes and compounds (e.g., substrates and the like) traditionally associated with the pantothenate biosynthetic pathway utilized in the formation or synthesis of HMBPA. The term “HMBPA biosynthetic pathway” includes the biosynthetic pathway leading to the synthesis of HMBPA in microorganisms (e.g., in vivo) as well as the biosynthetic pathway leading to the synthesis of HMBPA in vitro.

The term “HMBPA biosynthetic enzyme” includes any enzyme utilized in the formation of a compound (e.g., intermediate or product) of the HMBPA biosynthetic pathway. For example, synthesis of 2-hydroxyisovaleric acid (α-HIV) from α-ketoisovalerate (α-KIV) is catalyzed by the panE1 or panE2 gene product (PanE1, alternatively referred to herein ketopantoate reductase or PanE2, a α-ketoacid reductase that does not significantly contribute to pantothenate biosynthesis) and/or is catalyzed by the ilvC gene product (alternatively referred to herein as acetohydroxyacid isomeroreductase). Formation of HMBPA from β-alanine and α-HIV is catalyzed by the panC gene product (alternatively referred to herein as pantothenate synthetase).

The term “3-(2-hydroxy-3-methyl-butyrylamino)-propionic acid (“HMBPA”)” includes the free acid form of HMBPA, also referred to as “3-(2-hydroxy-3-methyl-butyrylamino)-propionate” as well as any salt thereof (e.g., derived by replacing the acidic hydrogen of [R]-3-(2-hydroxy-3-methyl-butyrylamino)-propionic acid or [R]-3-(2-hydroxy-3-methyl-butyrylamino)-propionate with a cation, for example, calcium, sodium, potassium, ammonium), also referred to as a “3-(2-hydroxy-3-methyl-butyrylamino)-propionic acid salt” or “HMBPA salt”. Preferred HMBPA salts are calcium HMBPA or sodium HMBPA. HMBPA salts of the present invention include salts prepared via conventional methods from the free acids described herein. An HMBPA salt of the present invention can likewise be converted to a free acid form of [R]-3-(2-hydroxy-3-methyl-butyrylamino)-propionic acid or [R]-3-(2-hydroxy-3-methyl-butyrylamino)-propionate by conventional methodology.

Various aspects of the invention are described in further detail in the following subsections.

I. Targeting Genes Encoding Various Pantothenate and/or Isoleucine-Valine(ilv) and/or HMBPA Biosynthetic Enzymes

In one embodiment, the present invention features targeting or modifying various biosynthetic enzymes of the pantothenate and/or isoleucine-valine(ilv) and/or HMBPA biosynthetic pathways. In particular, the invention features modifying various enzymatic activities associated with said pathways by modifying or altering the genes encoding said biosynthetic enzymes.

The term “gene”, as used herein, includes a nucleic acid molecule (e.g., a DNA molecule or segment thereof) that, in an organism, can be separated from another gene or other genes, by intergenic DNA (i.e., intervening or spacer DNA which naturally flanks the gene and/or separates genes in the chromosomal DNA of the organism). Alternatively, a gene may slightly overlap another gene (e.g., the 3′ end of a first gene overlapping the 5′ end of a second gene), said overlapping genes separated from other genes by intergenic DNA. A gene may direct synthesis of an enzyme or other protein molecule (e.g., may comprise coding seqeunces, for example, a contiguous open reading frame (ORF) which encodes a protein) or may itself be functional in the organism. A gene in an organism, may be clustered in an operon, as defined herein, said operon being separated from other genes and/or operons by the intergenic DNA. An “isolated gene”, as used herein, includes a gene which is essentially free of sequences which naturally flank the gene in the chromosomal DNA of the organism from which the gene is derived (i.e., is free of adjacent coding sequences which encode a second or distinct protein, adjacent structural sequences or the like) and optionally includes 5′ and 3′ regulatory sequences, for example promoter sequences and/or terminator sequences. In one embodiment, an isolated gene includes predominantly coding sequences for a protein (e.g., sequences which encode Bacillus proteins). In another embodiment, an isolated gene includes coding sequences for a protein (e.g., for a Bacillus protein) and adjacent 5′ and/or 3′ regulatory sequences from the chromosomal DNA of the organism from which the gene is derived (e.g., adjacent 5′ and/or 3′ Bacillus regulatory sequences). Preferably, an isolated gene contains less than about 10 kb, 5 kb, 2 kb, 1 kb, 0.5 kb, 0.2 kb, 0.1 kb, 50 bp, 25 bp or 10 bp of nucleotide sequences that naturally flank the gene in the chromosomal DNA of the organism from which the gene is derived.

The term “operon” includes at least two adjacent genes or ORFs, optionally overlapping in sequence at either the 5′ or 3′ end of at least one gene or ORF. The term “operon” includes a coordinated unit of gene expression that contains a promoter and possibly a regulatory element associated with one or more adjacent genes or ORFs (e.g., structural genes encoding enzymes, for example, biosynthetic enzymes). Expression of the genes (e.g., structural genes) can be coordinately regulated, for example, by regulatory proteins binding to the regulatory element or by anti-termination of transcription. The genes of an operon (e.g., structural genes) can be transcribed to give a single mRNA that encodes all of the proteins.

A “gene having a mutation” or “mutant gene” as used herein, includes a gene having a nucleotide sequence which includes at least one alteration (e.g., substitution, insertion, deletion) such that the polypeptide or protein encoded by said mutant exhibits an activity that differs from the polypeptide or protein encoded by the wild-type nucleic acid molecule or gene. In one embodiment, a gene having a mutation or mutant gene encodes a polypeptide or protein having an increased activity as compared to the polypeptide or protein encoded by the wild-type gene, for example, when assayed under similar conditions (e.g., assayed in microorganisms cultured at the same temperature). As used herein, an “increased activity” or “increased enzymatic activity” is one that is at least 5% greater than that of the polypeptide or protein encoded by the wild-type nucleic acid molecule or gene, preferably at least 5-10% greater, more preferably at least 10-25% greater and even more preferably at least 25-50%, 50-75% or 75-100% greater than that of the polypeptide or protein encoded by the wild-type nucleic acid molecule or gene. Ranges intermediate to the above-recited values, e.g., 75-85%, 85-90%, 90-95%, are also intended to be encompassed by the present invention. As used herein, an “increased activity” or “increased enzymatic activity” can also include an activity that is at least 1.25-fold greater than the activity of the polypeptide or protein encoded by the wild-type gene, preferably at least 1.5-fold greater, more preferably at least 2-fold greater and even more preferably at least 3-fold, 4-fold, 5-fold, 10-fold, 20fold, 50-fold, 100-fold or greater than the activity of the polypeptide or protein encoded by the wild-type gene.

In another embodiment, a gene having a mutation or mutant gene encodes a polypeptide or protein having a reduced activity as compared to the polypeptide or protein encoded by the wild-type gene, for example, when assayed under similar conditions (e.g., assayed in microorganisms cultured at the same temperature). A mutant gene also can encode no polypeptide or have a reduced level of production of the wild-type polypeptide. As used herein, a “reduced activity” or “reduced enzymatic activity” is one that is at least 5% less than that of the polypeptide or protein encoded by the wild-type nucleic acid molecule or gene, preferably at least 5-10% less, more preferably at least 10-25% less and even more preferably at least 25-50%, 50-75% or 75-100% less than that of the polypeptide or protein encoded by the wild-type nucleic acid molecule or gene. Ranges intermediate to the above-recited values, e.g., 75-85%, 85-90%, 90-95%, are also intended to be encompassed by the present invention. As used herein, a “reduced activity” or “reduced enzymatic activity” can also include an activity that has been deleted or “knocked out” (e.g., approximately 100% less activity than that of the polypeptide or protein encoded by the wild-type nucleic acid molecule or gene).

Activity can be determined according to any well accepted assay for measuring activity of a particular protein of interest. Activity can be measured or assayed directly, for example, measuring an activity of a protein isolated or purified from a cell or mocroorganism. Alternatively, an activity can be measured or assayed within a cell or mocroorganism or in an extracellular medium. For example, assaying for a mutant gene (i.e., said mutant encoding a reduced enzymatic activity) can be accomplished by expressing the mutated gene in a microorganism, for example, a mutant microorganism in which the enzyme is temperature-sensitive, and assaying the mutant gene for the ability to complement a temperature sensitive (Ts) mutant for enzymatic activity. A mutant gene that encodes an “increased enzymatic activity” can be one that complements the Ts mutant more effectively than, for example, a corresponding wild-type gene. A mutant gene that encodes a “reduced enzymatic activity” is one that complements the Ts mutant less effectively than, for example, a corresponding wild-type gene.

It will be appreciated by the skilled artisan that even a single substitution in a nucleic acid or gene sequence (e.g., a base substitution that encodes an amino acid change in the corresponding amino acid sequence) can dramatically affect the activity of an encoded polypeptide or protein as compared to the corresponding wild-type polypeptide or protein. A mutant gene (e.g., encoding a mutant polypeptide or protein), as defined herein, is readily distinguishable from a nucleic acid or gene encoding a protein homologue in that a mutant gene encodes a protein or polypeptide having an altered activity, optionally observable as a different or distinct phenotype in a microorganism expressing said mutant gene or producing said mutant protein or polypeptide (i.e., a mutant microorganism) as compared to a corresponding microorganism expressing the wild-type gene. By contrast, a protein homologue has an identical or substantially similar activity, optionally phenotypically indiscernable when produced in a microorganism, as compared to a corresponding microorganism expressing the wild-type gene. Accordingly it is not, for example, the degree of sequence identity between nucleic acid molecules, genes, protein or polypeptides that serves to distinguish between homologues and mutants, rather it is the activity of the encoded protein or polypeptide that distinguishes between homologues and mutants: homologues having, for example, low (e.g., 30-50% sequence identity) sequence identity yet having substantially equivalent functional activities, and mutants, for example sharing 99% sequence identity yet having dramatically different or altered functional activities.

It will also be appreciated by the skilled artisan that nucleic acid molecules, genes, protein or polypeptides for use in the instant invention can be derived from any microorganisms having a HMBPA biosynthetic pathway, an ilv biosynthetic pathway or a pantothenate biosynthetic pathway. Such nucleic acid molecules, genes, protein or polypeptides can be identified by the skilled artisan using Blown techniques such as homology screening, sequence comparison and the like, and can be modified by the skilled artisan in such a way that expression or production of these nucleic acid molecules, genes, protein or polypeptides occurs in a recombinant microorganism (e.g., by using appropriate promoters, ribosomal binding sites, expression or integration vectors, modifying the sequence of the genes such that the transcription is increased (taking into account the preferable codon usage), etc., according to techniques described herein and those known in the art).

In one embodiment, the genes of the present invention are derived from a Gram positive microorganism organism (e.g., a microorganism which retains basic dye, for example, crystal violet, due to the presence of a Gram-positive wall surrounding the microorganism). The term “derived from” (e.g., “derived from” a Gram positive microorganism) refers to a gene which is naturally found in the microorganism (e.g., is naturally found in a Gram positive microorganism). In a preferred embodiment, the genes of the present invention are derived from a microorganism belonging to a genus selected from the group consisting of Bacillus, Cornyebacterium (e.g., Cornyebacterium glutamicum), Lactobacillus, Lactococci and Streptomyces. In a more preferred embodiment, the genes of the present invention are derived from a microorganism is of the genus Bacillus. In another preferred embodiment, the genes of the present invention are derived from a microorganism selected from the group consisting of Bacillus subtilis, Bacillus lentimorbus, Bacillus lentus, Bacillus firmus, Bacillus pantothenticus, Bacillus amyloliquefaciens, Bacillus cereus, Bacillus circulans, Bacillus coagulans, Bacillus licheniformis, Bacillus megaterium, Bacillus pumilus, Bacillus thuringiensis, Bacillus halodurans, and other Group 1 Bacillus species, for example, as characterized by 16S rRNA type. In another preferred embodiment, the gene is derived from Bacillus brevis or Bacillus stearothermophilus. In another preferred embodiment, the genes of the present invention are derived from a microorganism selected from the group consisting of Bacillus licheniformis, Bacillus amyloliquefaciens, Bacillus subtilis, and Bacillus pumilus. In a particularly preferred embodiment, the gene is derived from Bacillus subtilis (e.g., is Bacillus subtilis-derived). The term “derived from Bacillus subtilis” or “Bacillus's subtilis-derived” includes a gene which is naturally found in the microorganism Bacillus subtilis. Included within the scope of the present invention are Bacillus-derived genes (e.g., B. subtilis-derived genes), for example, Bacillus or B. subtilis coaX genes, serA genes, glyA genes, coaA genes, pan genes and/or ilv genes.

In another embodiment, the genes of the present invention are derived from a Gram negative (excludes basic dye) microorganism. In a preferred embodiment, the genes of the present invention are derived from a microorganism belonging to a genus selected from the group consisting of Salmonella (e.g., Salmonella typhimurium), Escherichia, Klebsiella, Serratia, and Proteus. In a more preferred embodiment, the genes of the present invention are derived from a microorganism of the genus Escherichia. In an even more preferred embodiment, the genes of the present invention are derived from Escherichia coli. In another embodiment, the genes of the present invention are derived from Saccharomyces (e.g., Saccharomyces cerevisiae).

II. Recombinant Nucleic Acid Molecules and Vectors

The present invention further features recombinant nucleic acid molecules (e.g., recombinant DNA molecules) that include genes described herein (e.g., isolated genes), preferably Bacillus genes, more preferably Bacillus subtilis genes, even more preferably Bacillus subtilis pantothenate biosynthetic genes and/or isoleucine-valine (ilv) biosynthetic genes and/or HMBPA biosynthetic genes. The term “recombinant nucleic acid molecule” includes a nucleic acid molecule (e.g., a DNA molecule) that has been altered, modified or engineered such that it differs in nucleotide sequence from the native or natural nucleic acid molecule from which the recombinant nucleic acid molecule was derived (e.g., by addition, deletion or substitution of one or more nucleotides). Preferably, a recombinant nucleic acid molecule (e.g., a recombinant DNA molecule) includes an isolated gene of the present invention operably linked to regulatory sequences. The phrase “operably liked to regulatory sequence(s)” means that the nucleotide sequence of the gene of interest is linked to the regulatory sequence(s) in a manner which allows for expression (e.g., enhanced, increased, constitutive, basal, attenuated, decreased or repressed expression) of the gene, preferably expression of a gene product encoded by the gene (e.g., when the recombinant nucleic acid molecule is included in a recombinant vector, as defined herein, and is introduced into a microorganism).

The term “regulatory sequence” includes nucleic acid sequences which affect (e.g., modulate or regulate) expression of other nucleic acid sequences (i.e., genes). In one embodiment, a regulatory sequence is included in a recombinant nucleic acid molecule in a similar or identical position and/or orientation relative to a particular gene of interest as is observed for the regulatory sequence and gene of interest as it appears in nature, e.g., in a native position and/or orientation. For example, a gene of interest can be included in a recombinant nucleic acid molecule operably linked to a regulatory sequence which accompanies or is adjacent to the gene of interest in the natural organism (e.g., operably linked to “native” regulatory sequences (e.g., to the “native” promoter). Alternatively, a gene of interest can be included in a recombinant nucleic acid molecule operably linked to a regulatory sequence which accompanies or is adjacent to another (e.g., a different) gene in the natural organism. Alternatively, a gene of interest can be included in a recombinant nucleic acid molecule operably linked to a regulatory sequence from another organism. For example, regulatory sequences from other microbes (e.g., other bacterial regulatory sequences, bacteriophage regulatory sequences and the like) can be operably linked to a particular gene of interest.

In one embodiment, a regulatory sequence is a non-native or non-naturally-occurring sequence (e.g., a sequence which has been modified, mutated, substituted, derivatized, deleted including sequences which are chemically synthesized). Preferred regulatory sequences include promoters, enhancers, termination signals, anti-termination signals and other expression control elements (e.g., sequences to which repressors or inducers bind and/or binding sites for transcriptional and/or translational regulatory proteins, for example, in the transcribed mRNA). Such regulatory sequences are described, for example, in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. Regulatory sequences include those which direct constitutive expression of a nucleotide sequence in a microorganism (e.g., constitutive promoters and strong constitutive promoters), those which direct inducible expression of a nucleotide sequence in a microorganism (e.g., inducible promoters, for example, xylose inducible promoters) and those which attenuate or repress expression of a nucleotide sequence in a microorganism (e.g., attenuation signals or repressor sequences). It is also within the scope of the present invention to regulate expression of a gene of interest by removing or deleting regulatory sequences. For example, sequences involved in the negative regulation of transcription can be removed such that expression of a gene of interest is enhanced.

In one embodiment, a recombinant nucleic acid molecule of the present invention includes a nucleic acid sequence or gene that encode at least one bacterial gene product (e.g., a pantothenate biosynthetic enzyme, an isoleucine-valine biosynthetic enzyme and/or a HMBPA biosynthetic enzyme) operably linked to a promoter or promoter sequence. Preferred promoters of the present invention include Bacillus promoters and/or bacteriophage promoters (e.g., bacteriophage which infect Bacillus). In one embodiment, a promoter is a Bacillus promoter, preferably a strong Bacillus promoter (e.g., a promoter associated with a biochemical housekeeping gene in Bacillus or a promoter associated with a glycolytic pathway gene in Bacillus). In another embodiment, a promoter is a bacteriophage promoter. In a preferred embodiment, the promoter is from the bacteriophage SP01. In a particularly preferred embodiment, a promoter is selected from the group consisting of P₁₅, P₂₆ or P_{veg}, having for example, the following respective seqeunces: GCTATTGACGACAGCTATGGTTCACTGTCCACCAACCAAAACTGTGCTCAGT ACCGCCAATATTTCTCCCTTGAGGGGTACAAAGAGGTGTCCCTAGAAGAGAT CCACGCTGTGTAAAAATTTTACAAAAAGGTATTGACTTTCCCTACAGGGTGT GTAATAATTTAATTACAGGCGGGGGCAACCCCGCCTGT(SEQ ID NO:1), GCCTACCTAGCTTCCAAGAAAGATATCCTAACAGCACAAGAGCGGAAAGAT GTTTTGTTCTACATCCAGAACAACCTCTGCTAAAATTCCTGAAAAATTTTGCA AAAAGTTGTTGACTTTATCTACAAGGTGTGGTATAATAATCTTAACAACAGC AGGACGC (SEQ ID NO:2), and GAGGAATCATAGAATTTTGTCAAAATAATTTTATTGACAACGTCTTATTAAC GTTGATATAATTTAAATTTTATTTGACAAAAATGGGCTCGTGTTGTACAATA AATGTAGTGAGGTGGATGCAATG (SEQ ID NO:3). Additional preferred promoters include tef (the translational elongation factor (TEF) promoter) and pyc (the pyruvate carboxylase (PYC) promoter), which promote high level expression in Bacillus (e.g., Bacillus subtilis). Additional preferred promoters, for example, for use in Gram positive microorganisms include, but are not limited to, amy and SPO2 promoters. Additional preferred promoters, for example, for use in Gram negative microorganisms include, but are not limited to, cos, tac, trp, tei, trp-tet, lpp, lac, lpp-lac, lacIQ, T7, T5, T3, gal, trc, ara, SP6, λ-PR or λ-PL.

In another embodiment, a recombinant nucleic acid molecule of the present invention includes a terminator sequence or terminator sequences (e.g., transcription terminator sequences). The term “terminator sequences” includes regulatory sequences that serve to terminate transcription of mRNA. Terminator sequences (or tandem transcription terminators) can further serve to stabilize mRNA (e.g., by adding structure to mRNA), for example, against nucleases.

In yet another embodiment, a recombinant nucleic acid molecule of the present invention includes sequences which allow for detection of the vector containing said sequences (i.e., detectable and/or selectable markers), for example, genes that encode antibiotic resistance or sequences that overcome auxotrophic mutations, for example, trpC, fluorescent markers, drug markers, and/or calorimetric markers (e.g., lacZ/β-galactosidase). In yet another embodiment, a recombinant nucleic acid molecule of the present invention includes an artificial ribosome binding site (RBS) or a sequence that becomes transcribed into an artificial RBS. The term “artificial ribosome binding site (RBS)” includes a site within an mRNA molecule (e.g., coded within DNA) to which a ribosome binds (e.g., to initiate translation) which differs from a native RBS (e.g., a RBS found in a naturally-occurring gene) by at least one nucleotide. Preferred artificial RBSs include about 5-6, 7-8, 9-10, 11-12, 13-14, 15-16, 17-18, 19-20, 21-22, 23-24, 25-26, 27-28, 29-30 or more nucleotides of which about 1-2, 3-4, 5-6, 7-8, 9-10, 11-12, 13-15 or more differ from the native RBS (e.g., the native RBS of a gene of interest, for example, the native panB RBS TAAACATGAGGAGGAGAAAACATG (SEQ ID NO:4) or the native panD RBS ATTCGAGAAATGGAGAGAATATAATATG (SEQ ID NO:5)).

Preferably, nucleotides that differ are substituted such that they are identical to one or more nucleotides of an ideal RBS when optimally aligned for comparisons. Ideal RBSs include, but are not limited to, AGAAAGGAGGTGA (SEQ ID NO:6), TTAAGAAAGGAGGTGANNNNATG (SEQ ID NO:7), TTAGAAAGGAGGTGANNNNNATG (SEQ ID NO:8), AGAAAGGAGGTGANNNNNNNATG (SEQ ID NO:9), and AGAAAGGAGGTGANNNNNNATG (SEQ ID NO:10). Artificial RBSs can be used to replace the naturally-occurring or native RBSs associated with a particular gene. Artificial RBSs preferably increase translation of a particular gene. Preferred artificial RBSs (e.g., RBSs for increasing the translation of panB, for example, of B. subtilis panB) include CCCTCTAGAAGGAGGAGAAAACATG (SEQ ID NO:11) and CCCTCTAGAGGAGGAGAAAACATG (SEQ ID NO:12). Preferred artificial RBSs (e.g., RBSs for increasing the translation of panD, for example, of B. subtilis panD) include TTAGAAAGGAGGATTTAAATATG (SEQ ID NO:13), TTAGAAAGGAGGTTTAATTAATG (SEQ ID NO:14), TTAGAAAGGAGGTGATTTAAATG (SEQ ID NO:15), TTAGAAAGGAGGTGTTTAAAATG (SEQ ID NO:16), ATTCGAGAAAGGAGG TGAATATAATATG (SEQ ID NO:17), ATTCGAGAAAGGAGGTGAATAATAATG (SEQ ID NO:18), and ATTCGTAGAAAGGAGGTGAATTAATATG (SEQ ID NO:19).

The present invention further features vectors (e.g., recombinant vectors) that include nucleic acid molecules (e.g., genes or recombinant nucleic acid molecules comprising said genes) as described herein. The term “recombinant vector” includes a vector (e.g., plasmid, phage, phasmid, virus, cosmid or other purified nucleic acid vector) that has been altered, modified or engineered such that it contains greater, fewer or different nucleic acid sequences than those included in the native or natural nucleic acid molecule from which the recombinant vector was derived. Preferably, the recombinant vector includes a biosynythetic enzyme-encoding gene or recombinant nucleic acid molecule including said gene, operably linked to regulatory sequences, for example, promoter sequences, terminator sequences and/or artificial ribosome binding sites (RBSs), as defined herein. In another embodiment, a recombinant vector of the present invention includes sequences that enhance replication in bacteria (e.g., replication-enhancing sequences). In one embodiment, replication-enhancing sequences are derived from E. coli. In another embodiment, replication-enhancing sequences are derived from pBR322.

In yet another embodiment, a recombinant vector of the present invention includes antibiotic resistance sequences. The term “antibiotic resistance sequences” includes sequences which promote or confer resistance to antibiotics on the host organism (e.g., Bacillus). In one embodiment, the antibiotic resistance sequences are selected from the group consisting of cat (chloramphenicol resistance) sequences, tet (tetracycline resistance) sequences, erm (erythromycin resistance) sequences, neo (neomycin resistance) sequences, kan (kanamycin resistance) and spec (spectinomycin resistance) sequences. Recombinant vectors of the present invention can further include homologous recombination sequences (e.g., sequences designed to allow recombination of the gene of interest into the chromosome of the host organism). For example, bpr, vpr, and/or amyE sequences can be used as homology targets for recombination into the host chromosome. It will further be appreciated by one of skill in the art that the design of a vector can be tailored depending on such factors as the choice of microorganism to be genetically engineered, the level of expression of gene product desired and the like.

IV. Recombinant Microorganisms

The present invention further features microorganisms, i.e., recombinant microorganisms, that include vectors or genes (e.g., wild-type and/or mutated genes) as described herein. As used herein, the term “recombinant microorganism” includes a microorganism (e.g., bacteria, yeast cell, fungal cell, etc.) that has been genetically altered, modified or engineered (e.g., genetically engineered) such that it exhibits an altered, modified or different genotype and/or phenotype (e.g., when the genetic modification affects coding nucleic acid sequences of the microorganism) as compared to the naturally-occurring microorganism from which it was derived.

In one embodiment, a recombinant microorganism of the present invention is a Gram positive organism (e.g., a microorganism which retains basic dye, for example, crystal violet, due to the presence of a Gram-positive wall surrounding the microorganism). In a preferred embodiment, the recombinant microorganism is a microorganism belonging to a genus selected from the group consisting of Bacillus, Cornyebacterium, Lactobacillus, Lactococci and Streptomyces. In a more preferred embodiment, the recombinant microorganism is of the genus Bacillus. In another preferred embodiment, the recombinant microorganism is selected from the group consisting of Bacillus subtilis, Bacillus lentimorbus, Bacillus lentus, Bacillus firmus, Bacillus pantothenticus, Bacillus amyloliquefaciens, Bacillus cereus, Bacillus circulans, Bacillus coagulans, Bacillus licheniformis, Bacillus megaterium, Bacillus pumilus, Bacillus thuringiensis, Bacillus halodurans, and other Group 1 Bacillus species, for example, as characterized by 16S rRNA type. In another preferred embodiment, the recombinant microorganism is Bacillus brevis or Bacillus stearothermophilis. In another preferred embodiment, the recombinant microorganism is selected from the group consisting of Bacillus licheniformis, Bacillus amyloliquefaciens, Bacillus subtilis, and Bacillus pumilus.

In another embodiment, the recombinant microorganism is a Gram negative (excludes basic dye) organism. In a preferred embodiment, the recombinant microorganism is a microorganism belonging to a genus selected from the group consisting of Salmonella, Escherichia, Klebsiella, Serratia, and Proteus. In a more preferred embodiment, the recombinant microorganism is of the genus Escherichia. In an even more preferred embodiment, the recombinant microorganism is Escherichia coli. In another embodiment, the recombinant microorganism is Saccharomyces (e.g., S. cerevisiae).

A preferred “recombinant” microorganism of the present invention is a microorganism having a deregulated pantothenate biosynthesis pathway or enzyme, a deregulated isoleucine-valine (ilv) biosynthetic pathway or enzyme and/or a deregulated HMBPA biosynthetic pathway or enzyme. The term “deregulated” or “deregulation” includes the alteration or modification of at least one gene in a microorganism that encodes an enzyme in a biosynthetic pathway, such that the level or activity of the biosynthetic enzyme in the microorganism is altered or modified. Preferably, at least one gene that encodes an enzyme in a biosynthetic pathway is altered or modified such that the gene product is enhanced or increased. The phrase “deregulated pathway” can also include a biosynthetic pathway in which more than one gene that encodes an enzyme in a biosynthetic pathway is altered or modified such that the level or activity of more than one biosynthetic enzyme is altered or modified. The ability to “deregulate” a pathway (e.g., to simultaneously deregulate more than one gene in a given biosynthetic pathway) in a microorganism in some cases arises from the particular phenomenon of microorganisms in which more than one enzyme (e.g., two or three biosynthetic enzymes) are encoded by genes occurring adjacent to one another on a contiguous piece of genetic material termed an “operon” (defined herein). Due to the coordinated regulation of genes included in an operon, alteration or modification of the single promoter and/or regulatory element can result in alteration or modification of the expression of each gene product encoded by the operon. Alteration or modification of the regulatory element can include, but is not limited to removing the endogenous promoter and/or regulatory element(s), adding strong promoters, inducible promoters or multiple promoters or removing regulatory sequences such that expression of the gene products is modified, modifying the chromosomal location of the operon, altering nucleic acid sequences adjacent to the operon or within the operon such as a ribosome binding site, increasing the copy number of the operon, modifying proteins (e.g., regulatory proteins, suppressors, enhancers, transcriptional activators and the like) involved in transcription of the operon and/or translation of the gene products of the operon, or any other conventional means of deregulating expression of genes routine in the art (including but not limited to use of antisense nucleic acid molecules, for example, to block expression of repressor proteins). Deregulation can also involve altering the coding region of one or more genes to yield, for example, an enzyme that is feedback resistant or has a higher or lower specific activity.

In another preferred embodiment, a recombinant microorganism is designed or engineered such that at least one pantothenate biosynthetic enzyme, at least one isoleucine-valine biosynthetic enzyme, and/or at least one HMBPA biosynthetic enzyme is overexpressed. The term “overexpressed” or “overexpression” includes expression of a gene product (e.g., a biosynthetic enzyme) at a level greater than that expressed prior to manipulation of the microorganism or in a comparable microorganism which has not been manipulated. In one embodiment, the microorganism can be genetically designed or engineered to overexpress a level of gene product greater than that expressed in a comparable microorganism which has not been engineered.

Genetic engineering can include, but is not limited to, altering or modifying regulatory sequences or sites associated with expression of a particular gene (e.g., by adding strong promoters, inducible promoters or multiple promoters or by removing regulatory sequences such that expression is constitutive), modifying the chromosomal location of a particular gene, altering nucleic acid sequences adjacent to a particular gene such as a ribosome binding site, increasing the copy number of a particular gene, modifying proteins (e.g., regulatory proteins, suppressors, enhancers, transcriptional activators and the like) involved in transcription of a particular gene and/or translation of a particular gene product, or any other conventional means of deregulating expression of a particular gene routine in the art (including but not limited to use of antisense nucleic acid molecules, for example, to block expression of repressor proteins). Genetic engineering can also include deletion of a gene, for example, to block a pathway or to remove a repressor. In embodiments featuring microorganisms having deleted genes, the skilled artisan will appreciate that at least low levels of certain compounds may be required to be present in or added to the culture medium in order that the viability of the microorganism is not compromised. Often, such low levels are present in complex culture media as routinely formulated. Moreover, in processes featuring culturing microorganisms having deleted genes cultured under conditions such that commercially or industrially attractive quantities of product are produced, it may be necessary to supplement culture media with slightly increased levels of certain compounds. For example, in processes featuring culturing a microorganism having a deleted panB gene, at least low levels of pantothenate must be present in the media, e.g., levels such as those found in routinely formulated complex media, whereas slightly increased levels of pantothenate may be added to the media in order to produce commercially or industrially attractive amounts of, for example, HMBPA. For example, 10-30 mg/L pantothenate can be added to the media in order to produce commercially or industrially attractive amounts of HMBPA.

In another embodiment, the microorganism can be physically or environmentally manipulated to overexpress a level of gene product greater than that expressed prior to manipulation of the microorganism or in a comparable microorganism which has not been manipulated. For example, a microorganism can be treated with or cultured in the presence of an agent known or suspected to increase transcription of a particular gene and/or translation of a particular gene product such that transcription and/or translation are enhanced or increased. Alternatively, a microorganism can be cultured at a temperature selected to increase transcription of a particular gene and/or translation of a particular gene product such that transcription and/or translation are enhanced or increased.

V. Culturing and Fermenting Recombinant Microorganisms

The term “culturing” includes maintaining and/or growing a living microorganism of the present invention (e.g., maintaining and/or growing a culture or strain). In one embodiment, a microorganism of the invention is cultured in liquid media. In another embodiment, a microorganism of the invention is cultured in solid media or semi-solid media. In a preferred embodiment, a microorganism of the invention is cultured in media (e.g., a sterile, liquid media) comprising nutrients essential or beneficial to the maintenance and/or growth of the microorganism (e.g., carbon sources or carbon substrate, for example carbohydrate, hydrocarbons, oils, fats, fatty acids, organic acids, and alcohols; nitrogen sources, for example, peptone, yeast extracts, meat extracts, malt extracts, urea, ammonium sulfate, ammonium chloride, ammonium nitrate and ammonium phosphate; phosphorus sources, for example, phosphoric acid, sodium and potassium salts thereof, trace elements, for example, magnesium, iron, manganese, calcium, copper, zinc, boron, molybdenum, and/or cobalt salts; as well as growth factors such as amino acids, vitamins, growth promoters and the like).

Preferably, microorganisms of the present invention are cultured under controlled pH. The term “controlled pH” includes any pH which results in production of the desired product (e.g., HMBPA). In one embodiment microorganisms are cultured at a pH of about 7. In another embodiment, microorganisms are cultured at a pH of between 6.0 and 8.5. The desired pH may be maintained by any number of methods known to those skilled in the art.

Also preferably, microorganisms of the present invention are cultured under controlled aeration. The term “controlled aeration” includes sufficient aeration (e.g., oxygen) to result in production of the desired product (e.g., HMBPA). In one embodiment, aeration is controlled by regulating oxygen levels in the culture, for example, by regulating the amount of oxygen dissolved in culture media. Preferably, aeration of the culture is controlled by agitating the culture. Agitation may be provided by a propeller or similar mechanical agitation equipment, by revolving or shaking the cuture vessel (e.g., tube or flask) or by various pumping equipment. Aeration may be further controlled by the passage of sterile air or oxygen through the medium (e.g., through the fermentation mixture). Also preferably, microorganisms of the present invention are cultured without excess foaming (e.g., via addition of antifoaming agents).

Moreover, microorganisms of the present invention can be cultured under controlled temperatures. The term “controlled temperature” includes any temperature which results in production of the desired product (e.g., HMBPA). In one embodiment, controlled temperatures include temperatures between 15° C. and 95° C. In another embodiment, controlled temperatures include temperatures between 15° C. and 70° C. Preferred temperatures are between 20° C. and 55° C., more preferably between 30° C. and 50° C.

Microorganisms can be cultured (e.g., maintained and/or grown) in liquid media and preferably are cultured, either continuously or intermittently, by conventional culturing methods such as standing culture, test tube culture, shaking culture (e.g., rotary shaking culture, shake flask culture, etc.), aeration spinner culture, or fermentation. In a preferred embodiment, the microorganisms are cultured in shake flasks. In a more preferred embodiment, the microorganisms are cultured in a fermentor (e.g., a fermentation process). Fermentation processes of the present invention include, but are not limited to, batch, fed-batch and continuous processes or methods of fermentation. The phrase “batch process” or “batch fermentation” refers to a closed system in which the composition of media, nutrients, supplemental additives and the like is set at the beginning of the fermentation and not subject to alteration during the fermentation, however, attempts may be made to control such factors as pH and oxygen concentration to prevent excess media acidification and/or microorganism death. The phrase “fed-batch process” or “fed-batch” fermentation refers to a batch fermentation with the exception that one or more substrates or supplements are added (e.g., added in increments or continuously) as the fermentation progresses. The phrase “continuous process” or “continuous fermentation” refers to a system in which a defined fermentation media is added continuously to a fermentor and an equal amount of used or “conditioned” media is simultaneously removed, preferably for recovery of the desired product (e.g., HMBPA). A variety of such processes have been developed and are well-known in the art.

The phrase “culturing under conditions such that a desired compound is produced” includes maintaining and/or growing microorganisms under conditions (e.g., temperature, pressure, pH, duration, etc.) appropriate or sufficient to obtain production of the desired compound or to obtain desired yields of the particular compound being produced. For example, culturing is continued for a time sufficient to produce the desired amount of a compound (e.g., HMBPA). Preferably, culturing is continued for a time sufficient to substantially reach suitable production of the compound (e.g., a time sufficient to reach a suitable concentration of HMBPA or suitable ratio of HMBPA:pantothenate). In one embodiment, culturing is continued for about 12 to 24 hours. In another embodiment, culturing is continued for about 24 to 36 hours, 36 to 48 hours, 48 to 72 hours, 72 to 96 hours, 96 to 120 hours, 120 to 144 hours, or greater than 144 hours. In yet another embodiment, microorganisms are cultured under conditions such that at least about 5 to 10 g/L of compound are produced in about 36 hours, at least about 10 to 20 g/L compound are produced in about 48 hours, or at least about 20 to 30 g/L compound in about 72 hours. In yet another embodiment, microorganisms are cultured under conditions such that at least a ratio of HMBPA:HMBPA+pantothenate of 1:10 is achieved (i.e., 10% HMBPA versus 90% pantothenate, for example, as determined by comparing the peaks when a sample of product is analyzed be HPLC), preferably such that at least a ratio of 2:10 is achieved (20% HMBPA versus 90% pantotheante), more preferably such that a ratio of at least 2.5:10 is achieved (25% HMBPA versus 75% pantotheante), more preferably at least 3:10 (30% HMBPA versus 70% pantotheante), 4:10 (40% HMBPA versus 60% pantotheante), 5:10 (50% HMBPA versus 50% pantotheante), 6:10 (60% HMBPA versus 40% pantotheante), 7:10 (70% HMBPA versus 30% pantotheante), 8:10 (80% HMBPA versus 20% pantotheante), 9:10 (90% HMBPA versus 10% pantotheante) or greater.

The methodology of the present invention can further include a step of recovering a desired compound (e.g., HMBPA). The term “recovering” a desired compound includes extracting, harvesting, isolating or purifying the compound from culture media. Recovering the compound can be performed according to any conventional isolation or purification methodology known in the art including, but not limited to, treatment with a conventional resin (e.g., anion or cation exchange resin, non/ionic adsorption resin, etc.), treatment with a conventional adsorbent (e.g., activated charcoal, silicic acid, silica gel, cellulose, alumina, etc.), alteration of pH, solvent extraction (e.g., with a conventional solvent such as an alcohol, ethyl acetate, hexane and the like), dialysis, filtration, concentration, crystallization, recrystallization, pH adjustment, lyophilization and the like. For example, a compound can be recovered from culture media by first removing the microorganisms from the culture. Media are then passed through or over a cation exchange resin to remove cations and then through or over an anion exchange resin to remove inorganic anions and organic acids having stronger acidities than the compound of interest. The resulting compound can subsequently be converted to a salt (e.g., a calcium salt) as described herein.

Preferably, a desired compound of the present invention is “extracted”, “isolated” or “purified” such that the resulting preparation is substantially free of other media components (e.g., free of media components and/or fermentation byproducts). The language “substantially free of other media components” includes preparations of the desired compound in which the compound is separated from media components or fermentation byproducts of the culture from which it is produced. In one embodiment, the preparation has greater than about 80% (by dry weight) of the desired compound (e.g., less than about 20% of other media components or fermentation byproducts), more preferably greater than about 90% of the desired compound (e.g., less than about 10% of other media components or fermentation byproducts), still more preferably greater than about 95% of the desired compound (e.g., less than about 5% of other media components or fermentation byproducts), and most preferably greater than about 98-99% desired compound (e.g., less than about 1-2% other media components or fermentation byproducts). When the desired compound has been derivatized to a salt, the compound is preferably further free of chemical contaminants associated with the formation of the salt. When the desired compound has been derivatized to an alcohol, the compound is preferably further free of chemical contaminants associated with the formation of the alcohol.

In an alternative embodiment, the desired compound is not purified from the microorganism, for example, when the microorganism is biologically non-hazardous (e.g., safe). For example, the entire culture (or culture supernatant) can be used as a source of product (e.g., crude product). In one embodiment, the culture (or culture supernatant) is used without modification. In another embodiment, the culture (or culture supernatant) is concentrated. In yet another embodiment, the culture (or culture supernatant) is dried or lyophilized.

Preferably, a production method of the present invention results in production of the desired compound at a significantly high yield. The phrase “significantly high yield” includes a level of production or yield which is sufficiently elevated or above what is usual for comparable production methods, for example, which is elevated to a level sufficient for commercial production of the desired product (e.g., production of the product at a commercially feasible cost). In one embodiment, the invention features a production method that includes culturing a recombinant microorganism under conditions such that the desired product (e.g., HMBPA) is produced at a level greater than 2 g/L. In another embodiment, the invention features a production method that includes culturing a recombinant microorganism under conditions such that the desired product (e.g., HMBPA) is produced at a level greater than 10 g/L. In another embodiment, the invention features a production method that includes culturing a recombinant microorganism under conditions such that the desired product (e.g., HMBPA) is produced at a level greater than 20 g/L. In yet another embodiment, the invention features a production method that includes culturing a recombinant microorganism under conditions such that the desired product (e.g., HMBPA) is produced at a level greater than 30 g/L. In yet another embodiment, the invention features a production method that includes culturing a recombinant microorganism under conditions such that the desired product (e.g., HMBPA) is produced at a level greater than 40 g/L. The invention further features a production method for producing the desired compound that involves culturing a recombinant microorganism under conditions such that a sufficiently elevated level of compound is produced within a commercially desirable period of time.

Depending on the biosynthetic enzyme or combination of biosynthetic enzymes manipulated, it may be desirable or necessary to provide (e.g., feed) microorganisms of the present invention at least one biosynthetic precursor such that the desired compound or compounds are produced. The term “biosynthetic precursor” or “precursor” includes an agent or compound which, when provided to, brought into contact with, or included in the culture medium of a microorganism, serves to enhance or increase biosynthesis of the desired product. In one embodiment, the biosynthetic precursor or precursor is aspartate. In another embodiment, the biosynthetic precursor or precursor is β-alanine. The amount of aspartate or β-alanine added is preferably an amount that results in a concentration in the culture medium sufficient to enhance productivity of the microorganism (e.g., a concentration sufficient to enhance production of HMBPA. The term “excess β-alanine” includes β-alanine levels increased or higher that those routinely utilized for culturing the microorganism in question. For example, culturing the Bacillus microorganisms described in the instant Examples is routinely done in the presence of about 0-5 g/L β-alanine. Accordingly, excess β-alanine levels can include levels of about 5-10 g/L or more preferably about 5-20 g/L β-alanine. Biosynthetic precursors of the present invention can be added in the form of a concentrated solution or suspension (e.g., in a suitable solvent such as water or buffer) or in the form of a solid (e.g., in the form of a powder). Moreover, biosynthetic precursors of the present invention can be added as a single aliquot, continuously or intermittently over a given period of time.

In yet another embodiment, the biosynthetic precursor is valine. In yet another embodiment, the biosynthetic precursor is α-ketoisovalerate. Preferably, valine or α-ketoisovalerate is added in an amount that results in a concentration in the medium sufficient for production of the desired product (e.g., HMBPA) to occur. The term “excess α-KIV” includes α-KIV levels increased or higher that those routinely utilized for culturing the microorganism in question. For example, culturing the Bacillus microorganisms described in the instant Examples can be done in the presence of about 0-5 g/L α-KIV. Accordingly, excess α-KIV levels can include levels of about 5-10 g/L, and more preferably about 5-20 g/L. The term “excess valine” includes valine levels increased or higher that those routinely utilized for culturing the microorganism in question. For example, culturing the Bacillus microorganisms described in the instant Examples is routinely done in the presence of about 0-0.5 g/L valine. Accordingly, excess valine levels can include levels of about 0.5-5 g/L, preferably about 5-20 g/L valine. Biosynthetic precursors are also referred to herein as “supplemental biosynthetic substrates”.

Moreover, certain aspects of the present invention include culturing microorganisms (e.g., recombinant microorganisms) under conditions of increased steady state glucose, decreased steady state dissolved oxygen and/or decreased serine. The term “increased steady state glucose” includes steady state glucose levels increased or higher that those routinely utilized for culturing the microorganism in question. For example, culturing the Bacillus microorganisms described in the instant Examples is routinely done in the presence of about 0.2-1.0 g/L steady state glucose. Accordingly, increased steady state glucose levels can include levels of about 1-2 g/l, about 2-5 g/l, and preferably about 5-20 g/L steady state glucose. The term “decreased steady state dissolved oxygen” includes steady state dissolved oxygen levels less or lower that those routinely utilized for culturing the microorganism in question and, for example, inversely correlates with increased steady state glucose levels. For example, culturing the Bacillus microorganisms described in the instant Examples is routinely done in the presence of about 10-30% dissolved oxygen. Accordingly, decreased steady state dissolved oxygen can include levels of about 0-10%, and preferably about 0-5% steady state dissolved oxygen. The term “reduced serine” includes serine levels within the lower range of those routinely utilized for culturing the microorganism in question. For example, culturing the Bacillus microorganisms described in the instant Examples is routinely done in the presence of about 0-0.5 g/L serine. Accordingly, reduced serine levels can include, for example, levels of 0-0.1 g/L serine.

Another aspect of the present invention includes biotransformation processes which feature the recombinant microorganisms described herein. The term “biotransformation process”, also referred to herein as “bioconversion processes”, includes biological processes which results in the production (e.g., transformation or conversion) of appropriate substrates and/or intermediate compounds into a desired product (e.g., HMBPA).

In one embodiment, the invention features a biotransformation process for the production of HMBPA comprising contacting a microorganism which overexpresses a reductase (e.g., overexpresses PanE1, PanE2 and/or IlvC) with appropriate substrates or precursors under conditions such that HMBPA is produced and recovering said HMBPA. In another embodiment, the invention features a biotransformation process for the production of HMBPA comprising contacting a microorganism which has a reduced or deleted PanB activity with appropriate substrates or precursors under conditions such that HMBPA is produced and recovering said HMBPA. In yet another embodiment, the invention features a biotransformation process for the production of HMBPA comprising contacting a microorganism which overexpresses at least one reductase and has a reduced or deleted PanB activity with appropriate substrates or precursors under conditions such that HMBPA is produced and recovering said HMBPA. Preferred recombinant microorganisms for carrying out the above-described biotransformations include the recombinant microorganisms described herein. In yet another embodiment, the invention features a biotransformation reaction that includes contacting αHIV and β-alanine with isolated or purified PanC under conditions such that HMBPA is produced. α-HIV can optionally be obtained by contacting α-KIV with purified or isolated reductase (e.g., PanE1, PanE2 and/or IlvC) and a source of reducing equivalent, for example, NADH. Conditions under which α-HIV or HMBPA are produced can include any conditions which result in the desired production of α-HIV or HMBPA, respectively. In yet another embodiment, the present invention includes a method of producing α-HIV that includes culturing a microorganism that overexpresses PanE1 and/or PanE2, and/or IlvC and has a reduced or deleted PanC or PanD (to reduce HMBPA or β-alanine sunthesis, respectively) under conditions such that α-HIV is produced.

The microorganism(s) and/or enzymes used in the biotransformation reactions are in a form allowing them to perform their intended function (e.g., producing a desired compound). The microorganisms can be whole cells, or can be only those portions of the cells necessary to obtain the desired end result. The microorganisms can be suspended (e.g., in an appropriate solution such as buffered solutions or media), rinsed (e.g., rinsed free of media from culturing the microorganism), acetone-dried, immobilized (e.g., with polyacrylamide gel or k-carrageenan or on synthetic supports, for example, beads, matrices and the like), fixed, cross-linked or permeablized (e.g., have permeablized membranes and/or walls such that compounds, for example, substrates, intermediates or products can more easily pass through said membrane or wall).

This invention is further illustrated by the following examples which should not be construed as limiting. The contents of all references, patents and published patent applications cited throughout this application are incorporated herein by reference.

### EXAMPLES

### Example I

### Discovery and Characterization of the [R]-3-(2-hydroxy-3-methyl-butyrylamino)-propionic acid (HMBPA) Biosynthetic Pathway

In developing Bacillus strains for the production of pantothenate, various genetic manipulations were made to enzymes involved in the pantothenate biosynthetic pathway and the isoleucine-valine (ilv) pathway (FIG. 1) as described in U.S. patent application Ser. No. 09/400,494 and U.S. patent application Ser. No. 09/667,569. For example, strains having a deregulated panBCD operon and/or having deregulated panE1 exhibited enhanced pantothenate production (when cultured in the presence of β-alanine and α-KIV). Strains further deregulated for ilvBNC and ilvD exhibited enhanced pantothenate production in the presence of only β-alanine. Moreover, it was possible to achieve β-alanine independence by further deregulating panD.

An exemplary strain is PA824, a tryptophan prototroph, Spec and Tet resistant, deregulated for panBCD at the panBCD locus, deregulated for panE1 at the panE1 locus (two genes in the B. subtilis genome are homologous to E. coli panE, panE1 and panE2, the former encoding the major ketopantoate reductase involved in pantothenate production, while panE2 does not contribute to pantothenate synthesis (U.S. patent application Ser. No. 09/400,494), deregulated for ilvD at the ilvD locus, overexpressing an ilvBNC cassette at the amyE locus, and overexpressing panD at the bpr locus.

The production of pantothenic acid by PA824 was investigated in 14 L fermentor vessels. The composition of the batch and feed media are as follows.

BATCH

| | MATERIAL | g/L (final) |
|---|---|---|
| 1 | Yeast extract | 10 |
| 2 | Na Glutamate | 5 |
| 3 | (NH₄)₂SO₄ | 8 |
| 4 | KH₂PO₄ | 5 |
| 5 | K₂HPO₄ | 7.6 |

Addded After Sterilization and Cool Down

| | | | |
|---|---|---|---|
| 1 | Glucose | 2.5 | |
| 2 | CaCl₂ | 0.1 | |
| 3 | MgCl₂ | 1 | |
| 4 | Sodium Citrate | 1 | |
| 5 | FeSO₄.7 H₂O | 0.01 | |
| 5 | SM-1000X | 1 | ml |

The final volume of the batch medium is 6 L. The trace element solution Sm-1000X has following composition: 0.15 g Na₂MoO₄.2H₂O, 2.5 g H₃BO₃, 0.7 g CoCl₂.6 H₂O, 0.25 g CuSO₄.5H₂O, 1.6 g MnCl₂.4H₂O, 0.3 g ZnSO₄.7H₂O are dissolved in water final volume 1L).

The batch medium was inoculated with 60 ml of shake flask PA824 culture (OD=10 in SVY medium: Difco Veal Infusion broth 25 g, Difco Yeast extract 5 g, Sodium Glutamate 5 g, (NH₄)₂SO₄ 2.7 g in 740 ml H₂O, autoclave; add 200 ml sterile 1 M K₂HPO₄ (pH 7) and 60 ml sterile 50% Glucose solution (final volume 1L)). The fermentation was run at 43° C. at an air flow rate of 12 L/min as a glucose limited fed batch. The initial batched glucose (2.5 g/L) was consumed during exponential growth). Afterwards glucose concentrations were maintained between 0.2-1 g/L by continuous feeding of FEED solution as follows.

FEED

| | MATERIAL | g/L (final) | |
|---|---|---|---|
| 1 | Glucose | 550 | |
| 2 | CaCl₂ | 0.1 | |
| 3 | SM-1000X | 3 | ml |

The variable feed rate pump was computer controlled and linked to the glucose concentration in the tank by an algorithm. In this example the total feeding was 6 L.

During fermentation the pH was set at 7.2. Control was achieved by pH measurements linked to computer control. The pH value was maintained by feeding either a 5% NH₃-solution or a 20% H₃PO₄-solution. NH₃ acts simultaneousely as a N-source for the fermentation. The dissolved oxygen concentration [pO₂] was set at 30% by regulation of the agitation and aeration rate. Foaming was controlled by addition of silicone oil. After the stop of the addition of the feed solution, in this example after 48 h, the fermentation was continued until the [pO₂] value reached 95%. Then the fermentation was stopped by killing the microorganism through sterilization for 30 min. The successful sterilization was proven by plating a sample of the fermentation broth on agar plates. The pantothenate titer in the fermentation broth was 21.7 g/L after sterilization and removal of the cells by centrifugation (determined by HPLC analysis).

For HPLC analysis the fermentation broth sample was diluted with sterile water (1:40). 5 μl of this dilution was injected into a HPLC column (Aqua C18, 5 μm, 150*2.0 mm, Phenomenex™). Temperature of the column was held at 40° C. Mobile phase A was 14.8 mM H₃PO₃, mobile phase B 100% Acetonitrile. Flow rate was constant at 0.5 mL/min. A gradient was applied:

| | |
|---|---|
| start: | 2% mobile phase B |
| 0-3 min | linear increase to 3% mobile phase B |
| 3-3.5 min | linear increase to 20% mobile phase B |

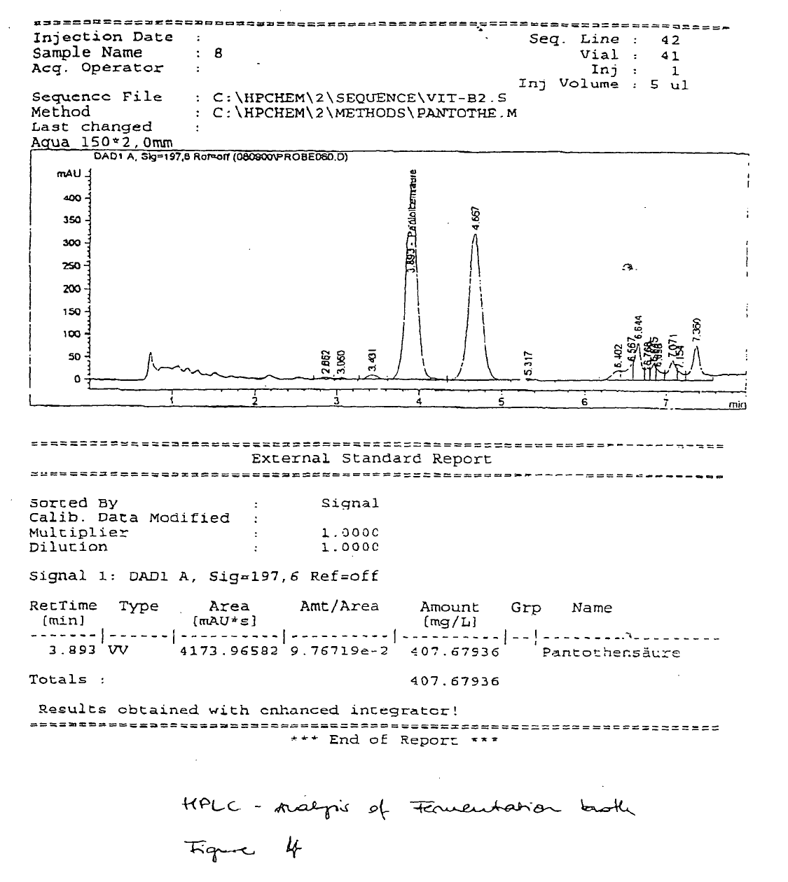

The detection was carried out by an UV-detector (210 nm). Run time was 7 min with an additional 3 min posttime. The retention time for pantothenic acid is 3.9 minutes. The HPLC chromatogram for the above mentioned sample is given in FIG. 4.

Identification of Compound Related to Peak with Retention Time 4.7 Minutes

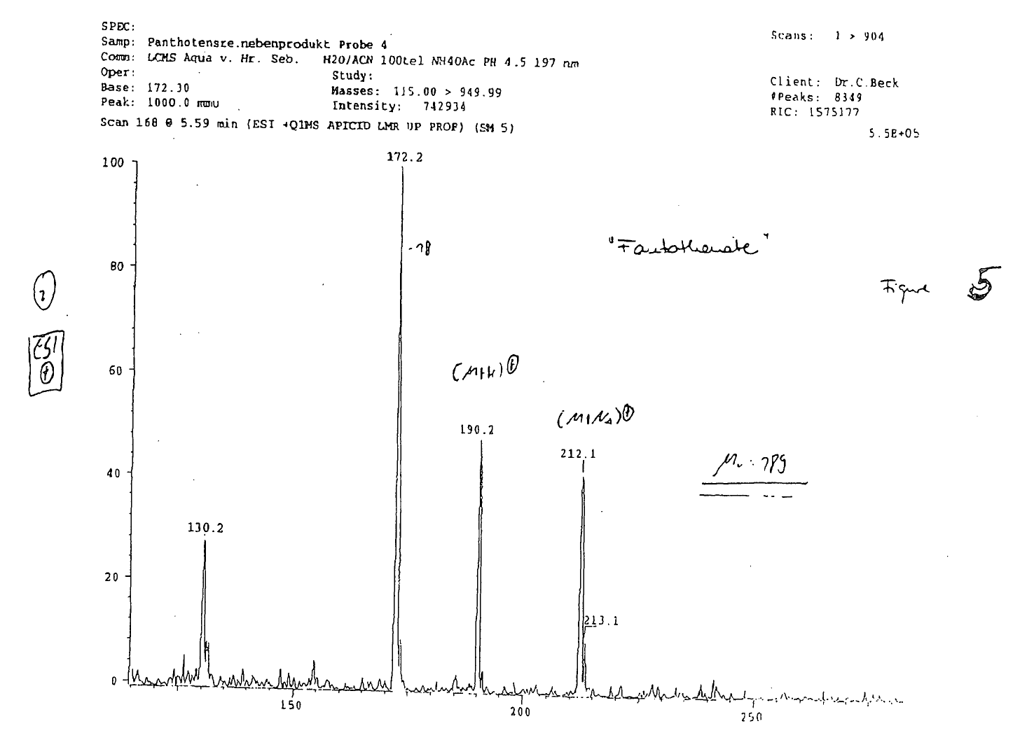

Under the described fermentation conditions, PA824 routinely yields approximately 20-30 g/L pantothenate. In addition to producing significant quantities of pantothenate, it was discovered a second compound eluted with an approximate retention time of 4.7 minutes in this system. The second prominent product formed in the fermentation was shown to be [R]-3-(2-hydroxy-3-methyl-butyrylamino)-propionic acid (HMBPA) (also referred to herein as “β-alanine 2-(R)-iydroxyisolvalerate”, “β-alanine 2-hydroxyisolvalerate”, and/or “β-alanyl-α-hydroxyisovalarate). It was identified by its mass spectrum (FIG. 5; relative monoisotopic mass 189), ¹H- and 13C-NMR (data not shown) after chromatographic purification by reverse phase flash chromatography (mobile phase 10 mM KH₂PO₄, with increasing contents of acetonitrile (1-50%)).

In order to verify the identity of the compound, deliberate synthesis of racemic β-alanine 2-hydroxyisolvalerate was performed as follows. β-alanine (2.73 g/30 mmol) and sodium methoxide (5.67 g of a 30% solution in methanol/31.5 mmol) were dissolved in methanol (40 mL). Methyl 2-hydroxyisovalerate (2-hydroxy-3-methylbutyric acid methyl ester) (3.96 g/30 mmol) was added and refluxed for 18 hours. Methanol was then removed by rotavap and replaced by tert-butanol (50 mL). Potassium tert-butoxide was added (50 mg) and refluxed for 26 hours. The solvent was removed in vacuo, the residue dissolved in water (50 mL) and passed through a strongly acidic ion-exchange resin (H+-form Lewatite™ S 100 G1; 100 mL). More water is used to rinse the ion exchanger. The aqueous eluates are combined and the water removed in vacuo. The residue is subjected to flash chromatography (silica gel; 2% acetic acid in ethyl acetate as eluent) and the product fractions evaporated to give a solid residue. The residue was recrystallized from ethyl acetate/toluene (10 mL /20 mL, respectively) and analytically pure HMBPA (β-alanine 2-hydroxyisolvalerate) was obtained, which showed a relative monoisotopic mass of 190 (189+H⁺) in the mass spec and the same ¹H-NMR resonances as the product obtained from fermentation.

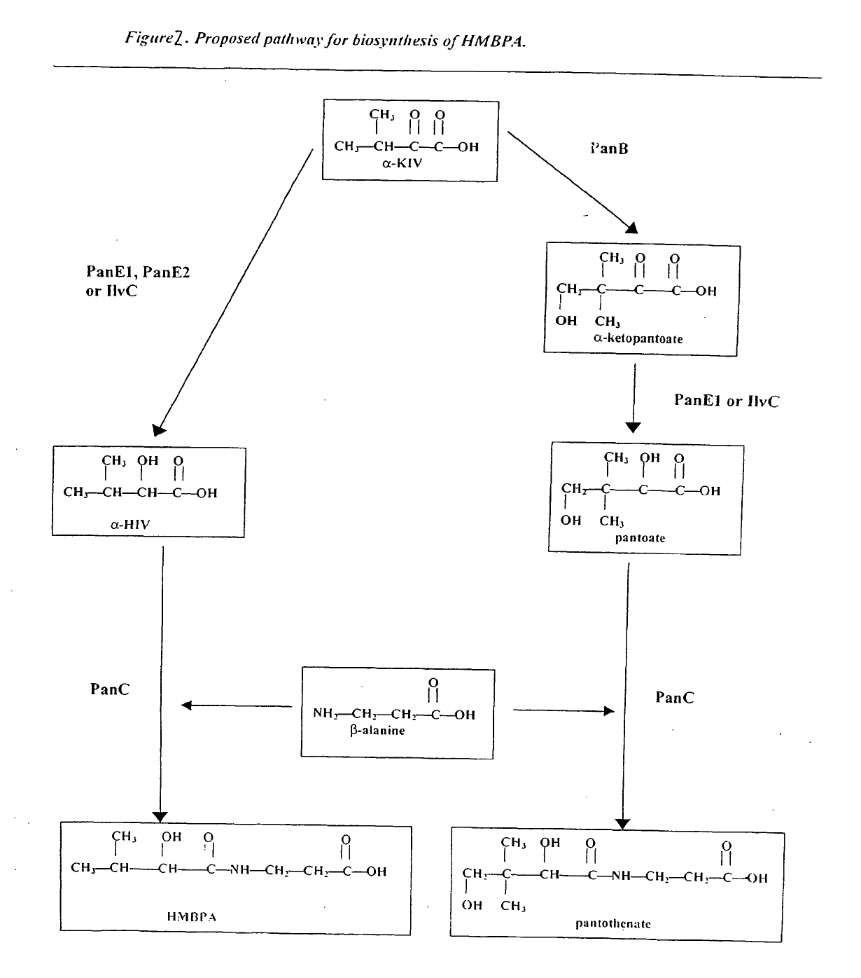

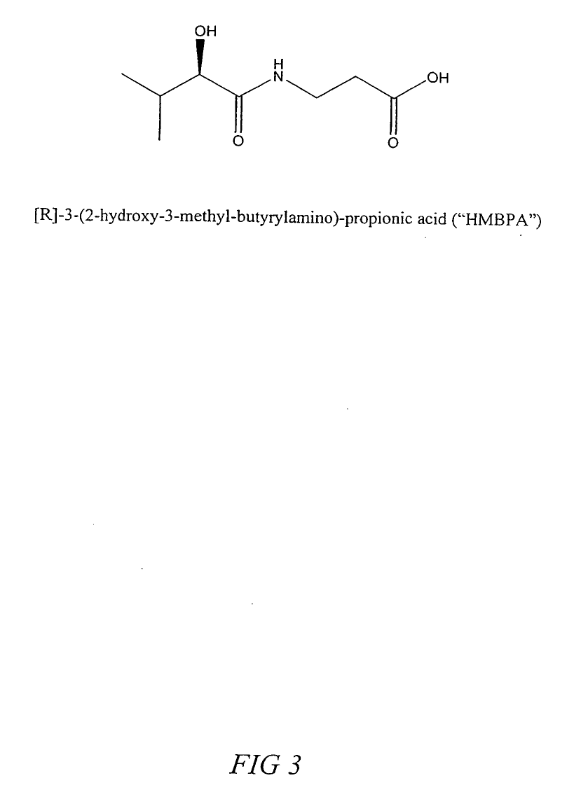

The biosynthetic pathway resulting in HMBPA production is set forth in FIG. 2. The chemical structure of [R]-3-(2-hydroxy-3-methyl-butyrylamino)propionic acid (HMBPA) is depicted in FIG. 3. As depicted in FIG. 2, HMBPA is the condensation product of α-hydroxyisovaleric acid (α-HIV) and β-alanine, catalyzed by the PanC enzyme. α-HIV is generated by reduction of α-KIV, a reaction which is catalyzed by the reductases PanE (e.g., PanE1 and/or PanE2) and/or IlvC.

Based on the chemical structure and biosynthetic pathway leading to HMBPA production, the present inventors formulated the following model to describe the interaction between the-previously known pantothenate and isoleucine-valine (ilv) pathways and the newly characterized HMBPA biosynthetic pathway. In at least one aspect, the model states that there exist at least two pathways in microorganisms that compete for α-KIV, the substrate for the biosynthetic enzyme PanB, namely the pantothenate biosynthetic pathway and the HMBPA biosynthetic pathway. (A third and fourth pathway competing for α-KIV are those resulting in the production of valine or leucine from α-KIV, see e.g., FIG. 1). At least the pantothenate biosynthetic pathway and the HMBPA biosynthetic pathway further produce competitive substrates for the enzyme PanC, namely α-HIV and pantoate. The model predicts that reducing PanB activity will increase α-KIV availability for α-HIV synthesis (and ultimately, HMBPA synthesis) and decrease the amount of pantoate and/or pantothenate synthesized by a microorganism. Conversely, increasing PanB activity will increase pantoate and ketopantoate availability for pantoate/pantothenate synthesis. The following examples provide experimental support for the model and further exemplify processes for increasing the production of HMBPA based on the model.

### Examples II-VI

For Examples II-VI, quanitation of pantothenate and/or HMBPA was performed as follows. Aliquots of fermentation media were diluted 1:100 and aliquots of test tube cultures were diluted 1:10 in water or 5% acetonitrile prior to injection on a Phenomenex Aqua™ 5μ C18 HPLC column (250×4.60 mm, 125A). Mobile phases were A=5% acetonitrile, 50 mM monosodium phosphate buffer adjusted to pH 2.5 with phosphoric acid; and B=95% acetonitrile, 5% H₂O.

Linear gradients were as follows.

| Minutes | Solvent A | Solvent B |
|---|---|---|
| 0 | 100% | 0% |
| 16 | 100% | 0% |
| 17 | 0% | 100% |
| 20 | 0% | 100% |
| 21 | 100% | 0% |

Additional parameters and apparatus were as follows: Flow rate=1.0 ml/min; Injection volume=20 μl; Detector=Hewlett Packard 1090 series DAD UV detector-3014, Signal A=197 nm, ref.=450 nm, Firmware revision E; Column heater=Oven tempature 40° C.; Hardware=Hewlett Packard Kayak™ XA; and Software=Hewlett Packard Chemstation Plus™ family revision A.06.03[509].

Under these fermentation conditions, PA824 routinely yields approximately 30-40 g/L pantothenate. HMBPA elutes at approximately 13 minutes in this system.

### Example II

### Ketopantoate Reductase Contributes to the Production of HMBPA and Increasing Ketopantoate Reductase Activity in Bacillus Results in Enhanced HMBPA Production

As described in Example I, a novel HPLC peak corresponding to HMBPA was observed in microorganisms overexpressing panE1 indicating that increased ketopantoate reductase contributes to the production of HMBPA (in addition to production of pantothenate). As mentioned previously, two genes in the B. subtilis genome are homologous to the E. coli panE gene encoding ketopantoate reductase and have been named panE1 and panE2. In Bacillus, the panE1 gene encodes the major ketopantoate reductase involved in pantothenate production, while panE2 does not contribute to pantothenate synthesis. In fact, overexpression of panE2 from a P₂₆ promoter leads to a reduction in pantothenate titer (see e.g., U.S. patent application Ser. No. 09/400,494).

Accordingly, it was tested whether, beside being produced by the panE1 gene product, it was possible that a significant portion of the α-HIV necessary to make HMBPA was being produced by the panE2 gene product. It was hypothesized that the panE2 gene product is an enzyme that can reduce α-KIV to α-HIV, but that can not significantly reduce ketopantoate to pantoate.

To test the hypothesis, panE2 was deleted from pantotheniate production strain PA824 (described in Example I) by transforming with a ΔpanE2::cat cassette from chromosomal DNA of strain PA248 (ΔpanE2::cat) (set forth as SEQ ID NO:24, for construction see e.g., U.S. patent application Ser. No. 09/400,494) to give strain PA919. Three isolates of PA919 were compared to PA824 for pantothenate and HMBPA production in test tube cultures grown in SVY plus β-alanine.

TABLE 1
Production of pantothenate and HMBPA by derivatives of PA824 and
PA880 grown at 43° C. in 48 hour test tube cultures
of SVY glucose + β-alanine⁵.

| Strain | new trait | parent | OD₆₀₀ | [pan ] g/l | [HMBPA] g/l |
|---|---|---|---|---|---|
| PA824 | — | | 13.9 | 4.3 | 0.64 |
| PA919-1 | ΔpanE2::cat | PA824 | 13.2 | 4.2 | 0.15 |
| PA919-2 | ″ | ″ | 14.8 | 3.8 | 0.13 |
| PA919-3 | ″ | ″ | 18.0 | 5.5 | 0.14 |

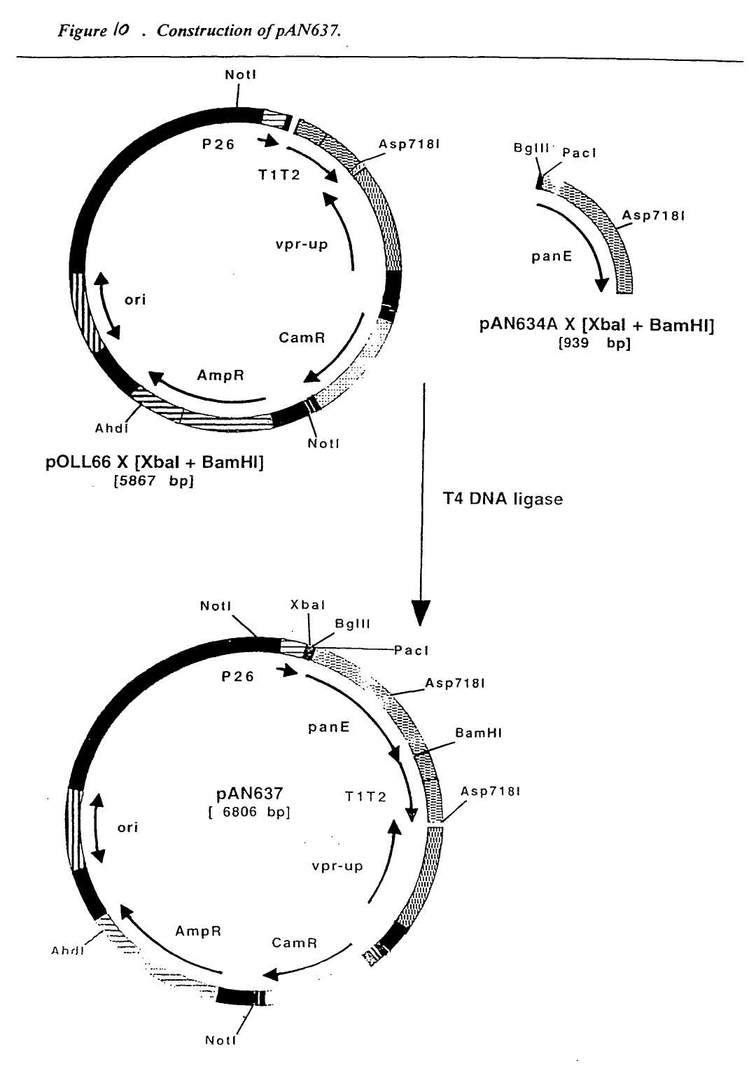

As indicated by the data in Table 1, all three isolates of PA919 produced about four-fold lower HMBPA than PA824 demonstrating that the panE2 gene product is a potent contributor to HMBPA synthesis. Moreover, significant increases in HMBPA production can be achieved simply by overexpression of panE2. An exemplary plasmid for the overexpression of panE2, named pAN238, is set forth as SEQ ID NO:25 (FIG. 10).

### Example III

### Increasing Production of HMBPA by Reducing PanB Activity in Microorganisms

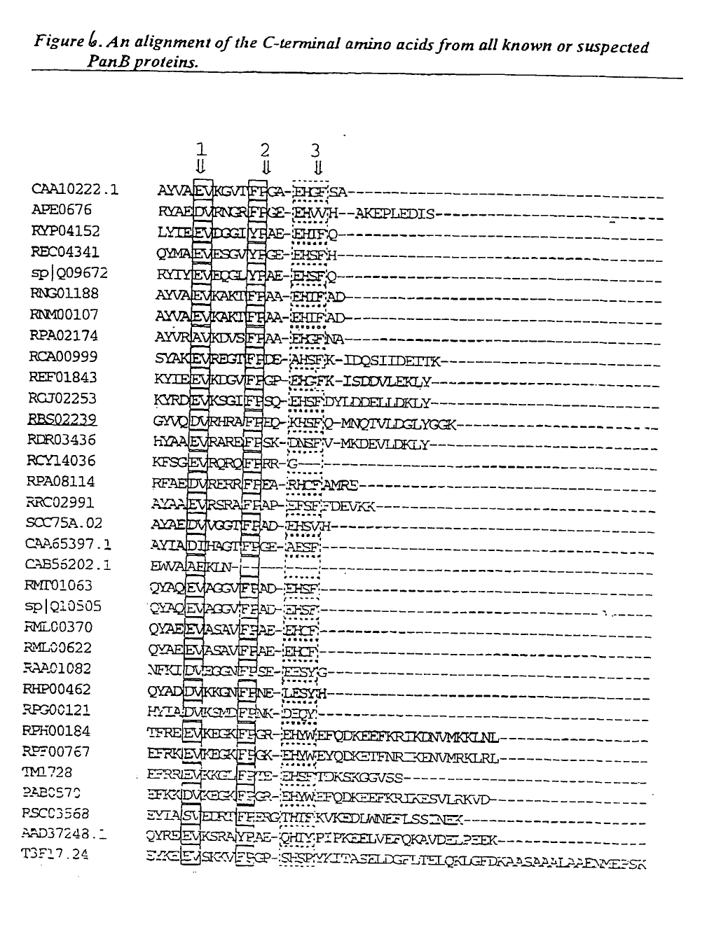

Strains derived from PA365 (the RL-1 lineage equivalent of PA377, described in U.S. patent application Ser. No. 09/667,569) which are deleted for the P₂₆ panBCD cassette and which contain a P₂₆panC*D cassette amplified at the vpr locus and either the wild type P₂₆panB cassette (PA666) or a P26 ΔpanB cassette (PA664) amplified at the bpr locus were constructed as follows. An alignment of the C-terminal amino acids of known or suspected PanB proteins is shown in FIG. 6. Three regions called 1, 2 and 3, that were identified having conserved or semi-conserved amino acid residues, are indicated by arrows at the top of the figure. The B. subtilis PanB protein (RBS02239) is underlined. Two of the PanB proteins (RCY14036 and CAB56202.1) are missing region 3 while the latter PanB protein is also missing region 2 and has non-conserved amino acid residues occupying region 1.

B. subtilis PanB variants were created that were missing regions 1, 2 and 3. The desired variants were created by designing 3′ PCR primers to amplify the B. subtilis pan B gene such that region 3, regions 2 and 3, or all three regions would be missing from the final product. The PCR products were generated and cloned into E. coli expression vector pASK-1BA3, creating plasmids pAN446, pAN447, and pAN448, respectively. The plasmids were then transformed into E. coli strain SJ2 that contains the panB6 mutation to test for complementation. Only pAN446, which is missing region 3, was able to complement. This indicates that region 3 is not essential for B. subtilis PanB activity but that region 2 is required for activity or stability.

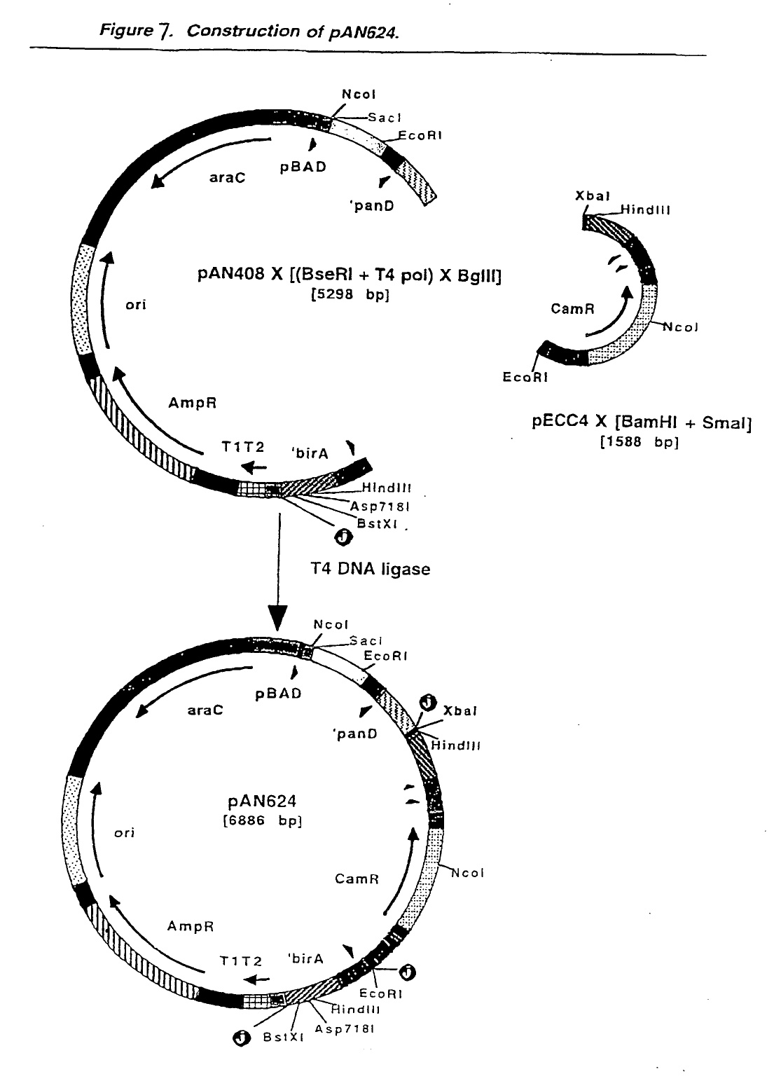

The next step in this analysis was to transfer the panB gene from pAN446 to a B. subtilis expression vector and then introduce it into a strain appropriate for testing activity of the encoded PanB protein in B. subtilis. To do this, a strain that is deleted for the P₂₆ panBCD operon was first created. This was accomplished by first inserting a cat gene between the BseRI site located just upstream of the panB RBS and the Bg/II site located in panD, creating plasmid pAN624 (FIG. 7). The sequence of pAN624 is set forth as SEQ ID NO:20. The resulting deletion-substitution mutation (ΔpanBCD::cat624), which removes all of panB and panC, was crossed into PA354 by transformation, with selection for resistance to chloramphenicol on plates supplemented with 1 mM pantothenate. One of the transformants was saved and named PA644. Chromosomal DNA isolated from PA644 was analyzed by PCR and was shown to contain the deletion-substitution mutation. As expected, PA644 requires pantothenate for growth but retains the engineered ilv genes (P₂₆ilvBNC P₂₆ilvD) as well as the P₂₆pan E1 gene originally present in PA354. Thus, it has all the enzymes involved in pantoate synthesis overproduced except PanB. The gene containing the shortest panB deletion was inserted into B. subtilis expression vector pOTP61 (described in U.S. patent application Ser. No. 09/667,569), creating plasmid pAN627. At the same time, a wild-type panB control gene was inserted into pOTP61, creating plasmid pAN630. The NotI fragments of each plasmid, lacking E. coli vector sequences, were ligated and transformed into PA644, with selection for resistance to tetracycline.

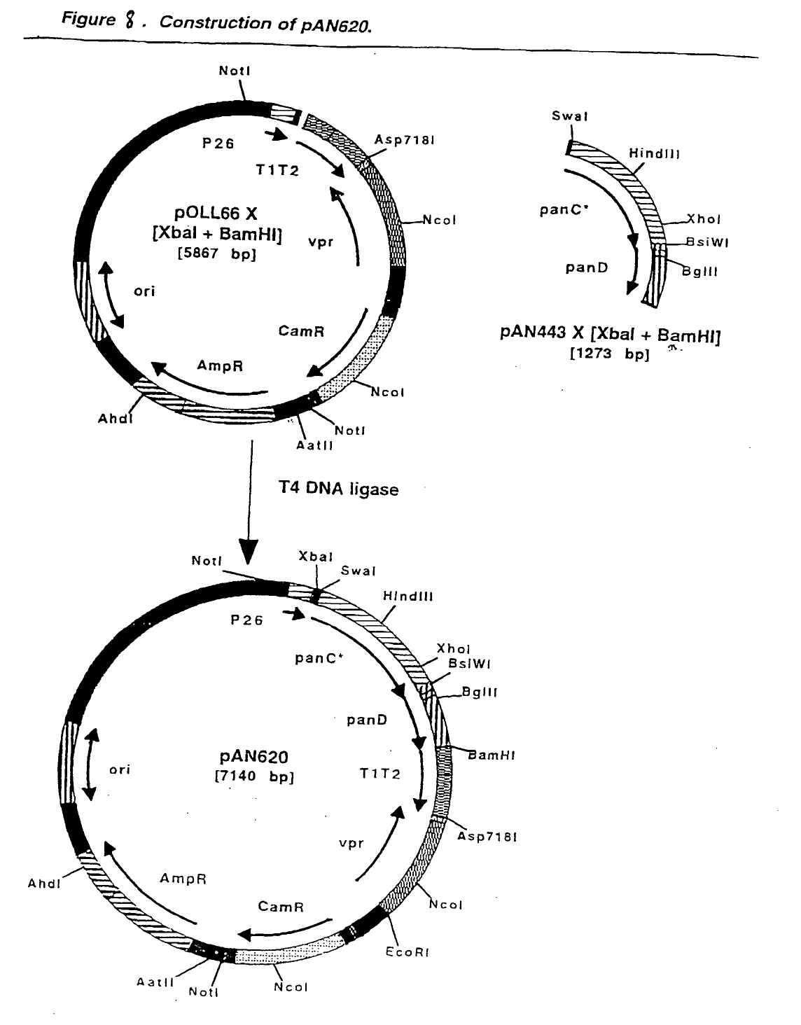

One transformant from each transformation was saved and further transformed with chromosomal DNA from PA628 with selection for Pan*. PA628 contains a multicopy P₂₆panC*D expression plasmid (pAN620) integrated at the vpr locus. In order to determine the effects of the panB gene mutation directly on pantothenate production, plasmid pAN620, set forth as SEQ ID NO:21 and illustrated schematically in FIG. 8, provides the remaining two enzymes required for pantothenate synthesis (PanC and PanD). Four transformants from each transformation were isolated, grown in SVY medium containing 10 g/L aspartate for 48 hours, then assayed for pantothenate production. Transformants with the 3′deleted panB gene were named PA664 and those containing the wild-type gene were called PA666. The data showed that the 3′ deleted panB gene in PA664 encodes a PanB protein with greatly reduced activity. To test for HMBPA production, test tube cultures of PA365, PA666, and PA664 were grown in SVY+aspartate medium with and without added α-KIV or pantoate for 48 hours and then assayed for HMBPA and pantothenate as described previously.

TABLE 2
Effect of PanB activity and addition of precursors on HMBPA and pantothenate production, 48 hour
test tube culture data, SVY + aspartate (10 g/L) medium.

| | | | | | | +α-KIV | | +pantoate | |
|---|---|---|---|---|---|---|---|---|---|
| | | | | no additions | | (5 g/L) | | (5 g/L) | |
| | | pan C*D | pauB | [pan] | HMBPA | [pan] | HMBPA | [pan] | HMBPA |
| Strain | pan operon | plasmid | plasmid | (g/L) | peak* | (g/L) | peak | (g/L) | peak |
| PA365 | P₂₆panBCD | NONE | NONE | 3.0 | 0.71 | 3.2 | 1.28 | 4.8 | 0.38 |
| PA666 | ΔpanBCD::cat | pAN620 | pAN630 | 3.7 | 0.55 | 3.3 | 1.70 | 5.2 | 0.26 |
| PA664 | ΔpanBCD::cat | pAN620 | pAN627 | 0.3 | 1.39 | 0.6 | 1.76 | 2.5 | 0.74 |

*HMBPA peak = peak area × 10⁻³

The data presented in Table 2 demonstrate that in the absence of supplements, PA664 produced the most HMBPA while PA666 produced the least, indicating an inverse correlation between PanB activity and HMBPA production. This is consistent with the model which predicts that the two pathways compete for α-KIV, the substrate for PanB, and produce competitive substrates for PanC; lowering PanB activity would be expected to increase α-KIV availability for α-HIV synthesis and increase HMBPA production, correspondingly decreasing the amount of pantoate synthesized. When α-KIV is added to the medium, all three strains produced significantly more HMBPA. This result evidences that α-KIV is a precursor to HMBPA, as described in FIG. 2, and that excess α-KIV favors HMBPA production. This result also suggests that synthesis of HMBPA is at least partially due to an overflow effect of excess α-KIV production. When pantoate was added to the medium, HMBPA was reduced by roughly 50 percent in all three strains. Conversely, the strains each produced significantly more pantothenate. This result is also consistent with the model that the two pathways produce competing substrates for PanC (α-HIV and pantoate). Taken together, the above results further indicate that decreasing pantoate synthesis should be beneficial in promoting HMBPA production as well as reducing pantothenate levels.

### Example IV

### Methods for Regulating HBPA:Pantothenate Levels

As demonstrated in Examples I and II, PanE1 and/or PanE2 contribute to enhanced HMBPA production as does reduced PanB activity. This Example demonstrates that overexpressing PanE1 increases HMBPA production relative to pantothenate production whereas overexpressing PanB decreases HMBPA production relative to pantothenate production. Furthermore, in strains overexpressing IlvC, HMBPA production is enhanced.

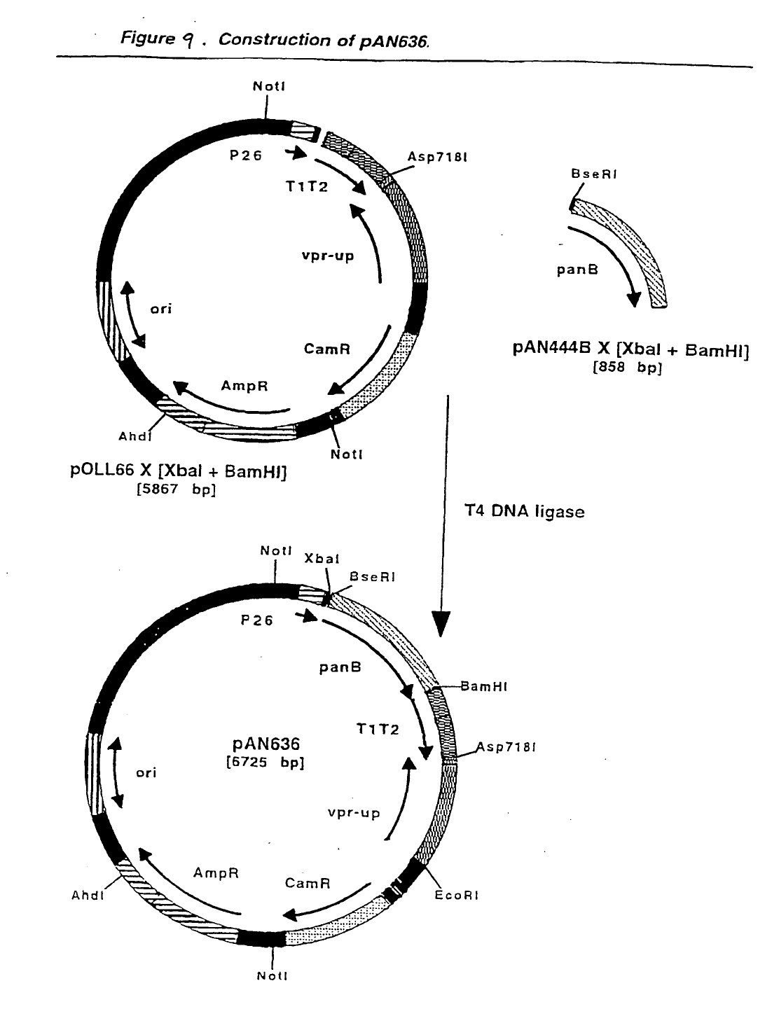

PA668 is a derivative of PA824 that contains extra copies of P₂₆panB amplified at the vpr or panB locus. PA668 was constructed using the panB expression vector (pAN636) which allows for selection of multiple copies using chloramphenicol. The sequence of pAN636 is set forth as SEQ ID NO:22 and the vector is depicted schematically in FIG. 9. The pAN636 NotI restriction fragment, missing the E. coli vector sequences, was ligated and then used to transform PA824 with selection on plates containing 5 μg/ml chloramphenicol. Transformants resistant to 30 μg/ml chloramphenicol were isolated and screened for pantothenate production in 48 hour test tube cultures. The isolates shown produce less HMBPA that PA824 (conversely producing about 10 percent more pantothenate than PA824). A second strain, called PA669, was constructed which is PA824 with extra copies of P26panE1 amplified at the vpr or panE1 locus. Strain PA669 was constructed by transforming PA824 with the self-ligated NotI fragment of plasmid pAN637 with selection for resistance to chloramphenicol. The sequence of pAN637 is set forth as SEQ ID NO:23 and the vector is depicted schematically in FIG. 10. Two isolates of PA669 were chosen for further study; PA669-5 produces less PanE1 than PA669-7 as judged by SDS-PAGE analysis of total cell extracts made from the two strains.

Test tube cultures of strains PA824, PA668-2, PA668-24, and the two isolates of PA669 (PA669-5 and PA669-7) were grown in three different media (SVY, SVY+aspartate, and SVY+aspartate +pantoate) for 48 hours and then assayed for pantothenate, HMBPA, and β-alanine (Table 3).

TABLE 3
Effect of extra copies of panB and panE1 on pantothenate and HMBPA production
by PA824, 48 hour test tube culture data, SV medium.

| | | | | | | | | | +aspartate (10 g/L) & | | |
|---|---|---|---|---|---|---|---|---|---|---|---|
| | | | no additions | | | +aspartate (10 g/L) | | | pantoate (5 g/L) | | |
| | panB | panE | [pan] | [β-ala] | HMBPA | [pan] | [β-ala] | | [pan] | [β-ala] | |
| Strain | plasmid | plasmid | (g/L) | (g/L) | * | (g/L) | (g/L) | HMBPA | (g/L) | (g/L) | HMBPA |
| PA824 | NONE | NONE | 1.8 | 0.05 | <0.1 | 4.7 | 2.5 | 0.53 | 5.6 | 2.5 | <0.10 |
| PA668-2 | pAN636 | NONE | 1.5 | <0.04 | <0.1 | 5.0 | 1.6 | <0.10 | 4.9 | 1.2 | <0.10 |
| PA668-24 | pAN636 | NONE | 1.8 | 0.05 | <0.1 | 4.9 | 2.8 | 0.34 | 6.1 | 2.6 | <0.10 |
| PA669-5 | NONE | pAN637 | 1.8 | 0.04 | <0.1 | 4.2 | 3.1 | 0.74 | 5.8 | 2.6 | 0.30 |
| PA669-7 | NONE | pAN637 | 1.8 | 0.06 | <0.1 | 3.7 | 3.2 | 1.41 | 5.2 | 2.5 | 0.75 |

*HMBPA = peak area × 10⁻³

None of the strains produced detectable quantities of HMBPA in SVY medium. All strains produced roughly equivalent amounts of pantothenate and low amounts of β-alanine indicating that β-alanine is limiting for both pantothenate and HMBPA synthesis in these cultures and that β-alanine is a precursor for both compounds. When grown in SVY+aspartate medium, the two PA669 isolates produced more HMBPA than PA824 whereas both PA668 isolates produced less HMBPA than PA824. It is noteworthy that the strain that produces the most PanE1 (PA669-7) produced the most HMBPA (and the least pantothenate). This suggests that high levels of PanE1 favor the production of HMBPA at the expense of lower pantothenate synthesis. It is also interesting that PA668-24 produced more HMBPA than PA668-2, even though SDS-PAGE analysis of extracts from the two strains showed that they produce roughly equivalent levels of PanB. The SDS-PAGE analysis also showed that PA668-24 makes much more IlvC than PA668-2. Based on these data, it is proposed that IlvC influences HMBPA synthesis by increasing steady state levels of α-KIV and/or by catalyzing α-HIV formation from α-KIV, thereby accounting for the observed shift towards production of HMBPA.

The final set of data in Table 3 shows that adding pantoate to the growth medium decreased HMBPA production by all strains that had previously produced detectable levels, e.g., by shifting synthesis towards pantothenate. This further supports the model that α-HIV and pantoate are competitive substrates for PanC.

### Example V

### Increasing HMBPA Production by Limiting Serine Availability

It was hypothesized that the ratio of pantothenate to HMBPA production could also be controlled by regulating the availability of serine or methylene tetrahydrofolate in the microorganism cultures. In particular, it is proposed that decreasing the availability of serine could increase HMBPA production relative to pantothenate production, whereas increasing the availability of serine would decrease the production of HMBPA relative to pantothenate production. This method is based on the understanding that the PanB substrate, methylenetetrahydrofolate is derived from serine. Thus, regulating serine levels should effectively regulate PanB substrate levels. To test this hypothesis, PA824 was grown in test tube cultures of SVY glucose plus 5 g/L β-alanine and ±5 g/L serine for 48 hours at 43° C.

TABLE 4
Production of HMBPA and pantothenate by PA824 with and without
the addition of serine

| serine added | | | |
|---|---|---|---|
| at 5 g/L | OD₆₀₀ | [pan] g/L | [HMBPA] g/L |
| − | 16.3 | 4.9 | 0.84 |
| − | 14.0 | 4.5 | 0.80 |
| + | 13.1 | 6.4 | 0.56 |
| + | 12.9 | 6.0 | 0.62 |

As demonstrated in Table 4, addition of serine decreases the level of production while conversely increasing pantothenate production. At least one method of decreasing methylene tetrahydrofolate levels in order to regulate HMBPA production levels is to decrease the activity of serine hydroxymethyl transferase (the glyA gene product), thereby decreasing methylene tetrahydrofolate biosynthesis in appropriately engineered microorganisms. At least one method of decreasing serine levels in order to regulate HMBPA production is to decrease the activity of 3-phosphoglycerate dehydrogenase (the serA gene product).

### Example VI

### Increasing HMBPA Production by Modifying Culture Conditions for Recombinant Microorganisms

In at least one fermentation (Fermentation P162), levels of HMBPA n reached 35 g/L. Briefly, fermentation of strain PA824 was carried out as in Example I but utilizing PFM-155 medium formulated as follows.

BATCH

| | MATERIAL | g/L (final) |
|---|---|---|
| 1 | Amberex 1003 | 5 |
| 2 | Cargill 200/20 (soy flour) | 40 |
| 3 | Na Glutamate | 5 |
| 4 | (NH₄)₂SO₄ | 8 |
| 5 | MgSO₄.7H₂O | 1 |
| 6 | MAZU DF204C | 1 |
| 7 | H₂O | qs to 4 L |

Added After Sterilization and Cool Down

| | | |
|---|---|---|
| 1 | KH₂PO₄ | 10 |
| 2 | K₂HPO₄.3H₂O | 20 |
| 3 | H₂O | qs to 400 ml |
| 1 | 80% Glucose | 20 |
| 2 | CaCl₂.2H₂O | 0.1 |
| 1 | Sodium Citrate | 1 |
| 2 | FeSO₄.7H₂O | 0.01 |
| 3 | SM-1000X | 1 X |

FEED

| | | |
|---|---|---|
| 1 | 80% Glucose | 800 |
| 2 | CaCl₂.2H₂O | 0.8 |
| 3 | H₂O | qs to 3500 ml |

Added After Sterilization and Cool Down

| | | |
|---|---|---|
| 1 | Sodium Citrate | 2.0 |
| 2 | FeSO₄.7H₂O | 0.02 |
| 3 | SM-1000X | 2 X |
| 4 | Glutamate Na | 5.0 |
| 5 | H₂O | qs to 500 ml |

However, as a result of loss of process control during the fermentation, the dissolved oxygen became limiting between 16 and 17 hours and glucose began to accumulate after 16 hours.

These changes in fermentation conditions produced the following significant results at or after 16 hours. Namely, synthesis of HMBPA began to increase with a corresponding decrease in pantothenate synthesis. In the four hour interval before 16 hours the culture produced 7 g/l HMBPA, four hours afterwards, 9.0 g/l. Pantothenate was the reverse with 10 g/l and 6.0 g/l produced between 12-16 hours and 16-20 hours, respectively. Between 20 and 36 hours the average rate of HMBPA synthesis was 1.0 gal hr. Overall, fermentation P162 produced 35 g/l of HMBPA in 36 hours.

Thus, it appears that overfeeding of glucose, and/or limitation of dissolved oxygen (e.g., beginning at about 16 hours) leads to an increase in HMBPA production. Accordingly, two methods for increasing HMBPA production (relative to pantothenate production) are to increase steady state glucose levels and/or decrease steady state dissolved oxygen levels.

Equivalents Those skilled in the art will recognize, or be able to ascertain using no more than routine experimentation, many equivalents to the specific embodiments of the invention described herein. Such equivalents are intended to be encompassed by the following claims.

What is claimed:

1. A process for the production of 3-(2-hydroxy-3-methyl-butyrylamino)-propionic acid (HMBPA), comprising culturing a microorganism under conditions such that HMBPA is produced and detecting the HMBPA produced by said microorganism.

2. A process for the production of 3-(2-hydroxy-3-methyl-butyrylamino)-propionic acid (HMBPA), comprising culturing a microorganism under conditions such that HMBPA is produced and isolating the HMBPA produced by said microorganism.

3. A process for the production of 3-(2-hydroxy-3-methyl-butyrylamino)-propionic acid (HMBPA), comprising culturing a microorganism having increased keto reductase activity or increased pantothenate synthetase activity in the presence of excess α-ketoisovalerate and excess β-alanine, such that HMBPA is produced.

4. A process for the production of 3-(2-hydroxy-3-methyl-butyrylamino)-propionic acid (HMBPA), comprising culturing a microorganism having increased keto reductase activity and increased pantothenate synthetase activity in the presence of excess α-ketoisovalerate and excess β-alanine, such that HMBPA is produced.

5. The process of claim 3 or 4, wherein said microorganism comprises a modified panE gene.

6. The process of claim 5, wherein the panE gene is overexpressed, deregulated or present in multiple copies.

7. The process of claim 3 or 4, wherein said microorganism comprises a modified panE1 gene.

8. The process of claim 3 or 4, wherein said microorganism comprises a modified panE2 gene.

9. The process of claim 3 or 4, wherein said microorganism comprises a modified panE1 gene and a modified panE2 gene.

10. The process of claim 3 or 4, wherein said microorganism comprises a modified panC gene.

11. The process of claim 3 or 4, wherein the panC gene is overexpressed, deregulated or present in multiple copies.

12. The process of claim 3 or 4, wherein said microorganism further has increased acetohydroxyacid isomeroreductase activity.

13. A process for the production of 3-(2-hydroxy-3-methyl-butyrylamino)-propionic acid (HMBPA), comprising culturing a microorganism having increased acetohydroxyacid isomeroreductase activity in the presence of excess α-ketoisovalerate and excess β-alanine, such that HMBPA is produced.

14. The process of claim 12 or 13, wherein said microorganism comprises a modified ilvC gene.

15. The process of claim 14, wherein the ilvC gene is overexpressed, deregulated or present in multiple copies.

16. The process of any one of claims 3, 4 or 11, wherein said microorganism further has reduced ketopantoate hydroxymethyltransferase activity.

17. The process of claim 16, wherein said microorganism comprises a modified panB gene.

18. The process of claim 16, wherein said microorganism has been deleted for the panB gene.

19. A process for the production of 3-(2-hydroxy-3-methyl-butyrylamino)-propionic acid (HMBPA), comprising culturing a microorganism having reduced ketopantoate hydroxymethyltransferase activity in the presence of excess α-ketoisovalerate and excess β-alanine, such that HMBPA is produced.

20. A method for enhancing production of 3-(2-hydroxy-3-methyl-butyrylamino)-propionic acid (HMBPA) relative to pantothenate, comprising culturing a recombinant microorganism under conditions such that the HMBPA production is enhanced relative to pantothenate production.

21. A process for the production of 2-hydroxyisovaleric acid (α-HIV), comprising culturing a microorganism which overexpresses PanE1 or PanE2 and which further has reduced PanC or PanD activity under conditions such that α-HIV is produced.

22. A process for the production of 3-(2-hydroxy-3-methyl-butyrylamino)-propionic acid (HMBPA), comprising culturing a recombinant microorganism having decreased expression or activity of serA or glyA under conditions such that HMBPA is produced.

23. A process for the production of 3-(2-hydroxy-3-methyl-butyrylamino)-propionic acid (HMBPA), comprising culturing a recombinant microorganism having decreased expression or activity of serA and galA under conditions such that HMBPA is produced.

24. The process of any one of the proceeding claims wherein the microorganism is cultured under conditions of increased steady state glucose.

25. The process of any one of the proceeding claims wherein the microorganism is cultured under conditions of decreased steady state dissolved oxygen.

26. The process of any one of the proceeding claims wherein the microorganism is cultured under conditions of decreased serine.

27. A product produced according to any one of the above claims.

28. A recombinant microorganism that produces 3-(2-hydroxy-3-methyl-butyrylamino)-propionic acid (HMBPA), the microorganism having a modification in at least one gene encoding ketopantoate reductase that results in increased reductase activity and having a mutation or deletion in the panB gene that results in reduced ketopantoate hydroxymethyltransferase activity.

29. The recombinant microorganism of claim 28, wherein the gene encoding ketopantoate reductase is a panE gene.

30. The recombinant microorganism of claim 29, wherein the panE gene is panE1.

31. The recombinant microorganism of claim 29, wherein the panE gene is panE2.

32. The recombinant microorganism of claim 28, wherein the microorganism has a modification in panE1 and panE2.

33. The recombinant microorganism of claim 28, further having a modification in ilvC that results in increased acetohydroxyacid isomeroreductase activity.

34. A recombinant microorganism that produces 3-(2-hydroxy-3-methyl-butyrylamino)-propionic acid (HMBPA), the microorganism having a modification in ilvC that results in increased acetohydroxyacid isomeroreductase activity and having a mutation or deletion in the panB gene that results in reduced ketopantoate hydroxymethyltransferase activity.

35. The recombinant microorganism of any one of claims 28 to 34, wherein said microorganism belongs to the genus Bacillus.

36. The recombinant microorganism of claim 35, which is Bacillus subtilis.
